(19) United States
(12) Patent Application Publication
(10) Pub. No.: US 2014/0287945 A1
(43) Pub. Date: Sep. 25, 2014

(54) SURFACE OXIDATION FOR SEQUESTERING BIOMOLECULES AND RELATED METHODS

(71) Applicant: NVS Technologies, Inc., Menlo Park, CA (US)

(72) Inventors: Aldrich N. K. Lau, Palo Alto, CA (US); Robert G. Eason, Los Gatos, CA (US); Kristian Scaboo, Castro Valley, CA (US)

(73) Assignee: NVS Technologies, Inc., Menlo Park, CA (US)

(21) Appl. No.: 14/212,471

(22) Filed: Mar. 14, 2014

Related U.S. Application Data
(60) Provisional application No. 61/785,987, filed on Mar. 14, 2013.

(51) Int. Cl. C12Q 1/68

(52) U.S. Cl. CPC C12Q 1/6837
USPC 506/9; 506/16; 506/18; 506/19; 506/32; 506/38; 526/245

(57) ABSTRACT

Solid supports comprising polymers covalently bound to a solid substrate are provided. The polymers find utility in any number of applications including immobilizing analyte molecules to solid supports for high throughput assays.

### STATEMENT OF GOVERNMENT INTEREST

Partial funding of the work described herein was provided by the U.S. Department of Homeland Security under Contract No. HSHQDC-10-C-00053. The U.S. Government has certain rights in this invention.

### BACKGROUND

1. Field of the Invention

The present invention is generally directed to polymers bound to oxidized surfaces, novel polymers and methods for use of the same.

2. Description of the Related Art

Bioassays are used to probe for the presence and/or quantity of an analyte material in a biological sample. In surface-based assays, such as DNA microarrays, the analyte species is generally captured and detected on a solid support or substrate. The use of DNA microarrays has become widely adopted in the study of gene expression and genotyping due to the ability to monitor large numbers of genes simultaneously (Schena et al., Science 270:467-470 (1995); Pollack et al., Nat. Genet. 23:41-46 (1999)). Surface arrays can also be fabricated using other binding moieties such as carbohydrates, antibodies, proteins, haptens or aptamers, in order to facilitate a wide variety of bioassays in array format.

An effective functionalized material for bioassay applications must have adequate capacity to immobilize a sufficient amount of an analyte from relevant samples in order to provide a suitable signal when subjected to detection (e.g., polymerase chain reaction). Suitable functionalized materials must also provide a highly reproducible surface in order to be gainfully applied to profiling experiments, particularly in assay formats in which the sample and the control must be analyzed on disparate support surfaces with which they are associated, e.g., different supports or different locations on the same support. For example, supports that are not based on a highly reproducible surface chemistry can result in significant errors when undertaking assays (e.g., profiling comparisons), due to variations from support to support or different locations on the same support.

Surface arrays (e.g., “DNA chips”) have been prepared by using polymers to attach the analyte to the solid support. In general, arrays that include a polymer are formed by the in situ polymerization of precursor monomers or prepolymers on a solid substrate (e.g., bead, particle, plate, etc.). The selectivity and reproducibility of arrays that include organic polymers is frequently highly dependent upon a number of experimental variables including, monomer concentration, monomer ratios, initiator concentration, solvent evaporation rate, ambient humidity (in the case when the solvent is water), crosslinker concentration, purity of the monomers/crosslinker/solvent, laboratory temperature, pipetting time, sparging conditions, reaction temperature (in the case of thermal polymerizations), reaction humidity, uniformity of ultraviolet radiation (in the case of UV photopolymerization) and ambient oxygen conditions. While many of these parameters can be controlled in a manufacturing setting, it is difficult if not impossible to control all of these parameters. As a result, in situ polymerization results in relatively poor reproducibility from spot-to-spot, chip-to-chip and lot-to-lot.

In addition, while a significant amount of work has been expended upon the development of array surfaces using silica based substrates, e.g., glass, quartz, fused silica, and silicon (See, e.g., D. Cuschin et al., Anal. Biochem. 1997, 250, 203-211; G. M. Harbers et al., Chem. Mater. 2007, 19, 4405-4414; and U.S. Pat. No. 6,790,613, to Shi et al., U.S. Pat. No. 5,932,711, to Boles et al., U.S. Pat. No. 6,994,972, to Bardhan, et al., U.S. Pat. No. 7,781,203, to Frutos et al., and U.S. Pat. No. 7,217,512 and U.S. Pat. No. 7,541,146 to Lewis et al.), certain advantages are derived from using less expensive, more easily manufactured substrates, such as polymeric substrates. However, additional challenges have been encountered both in the selection and preparation of such substrates for bioassay purposes. For example, polymeric substrates often suffer worse problems as a result of additional surface functionalization, such as increased auto fluorescence, increased hydrophobicity, as well as challenges in attaching or associating the in situ polymerized coating to the underlying polymer substrate.

Accordingly, while progress has been made in this field, there remains a need in the art for improved functionalized solid substrates, polymers and methods for attaching analytes to these solid substrates and solid supports comprising such polymers for use in various assays, such as DNA microarrays. The present invention fulfills this need and provides further related advantages.

### BRIEF SUMMARY

In brief, the present invention is generally directed to solid supports comprising polymers covalently bound to solid substrates. Optionally, the polymers may comprise a capture probe covalently bound thereto, or a functional group for use in formation of covalent bonds with capture probes. Thus the solid supports find utility in any number of applications, including immobilizing a capture probe on a solid substrate for use in analytical assays. Solid substrates comprising reactive groups suitable for reaction or interaction with the polymers, and solid supports comprising the polymers and optional capture probes are also provided. The presently disclosed polymers, solid substrates and solid supports are useful in a variety of analytical applications, for example DNA and protein microarrays for use in individual point of care situations (doctor's office, emergency room, home, in the field, etc.), high throughput testing and other applications.

The solid substrates generally comprise alcohol, carbonyl and/or amine moieties to which the polymers are bound. Accordingly, certain embodiments of the present invention provide advantages over previously described solid supports since the polymers can be covalently bound directly to the solid substrates (e.g., organic polymers) via the alcohol, carbonyl and/or amine moieties without an intervening “tie layer.”

The presently described polymers, solid substrates, solid supports and related methods provide a number of advantages in various embodiments. For example, in certain embodiments the reactive groups described herein for conjugating the polymers to the capture probe are substantially inert except under specific conditions provided during the conjugation reaction, insuring a predictable and optimal level of reactivity during the conjugation process. Some embodiments also employ “click” chemistry (e.g., reaction of azides and alkynes to form triazoles) for conjugating a polymer to a capture probe (e.g., biomolecule such as DNA or an oligonucleotide), and such chemistry is substantially pH-insensitive and produces limited or no reaction by-products.

Accordingly, in one embodiment the disclosure provides a solid support comprising:

a substrate having an outer surface; and

a plurality of polymers covalently bound to the outer surface of the substrate, the polymers each comprising at least one A and C subunit and optionally comprising one or more B subunits, wherein:

the A subunit, at each occurrence, independently comprises:
a) a first thermochemically reactive group, wherein the first thermochemically reactive group is capable of forming a covalent bond with an alcohol, carbonyl or amine group on a capture probe;
b) a second thermochemically reactive group, wherein the second thermochemically reactive group is cycloaddition or conjugate addition reactive group having a reactivity specific for covalent bond formation with a target functional group on a capture probe via a cycloaddition or 1,4-conjugate addition reaction; or
c) a covalent bond to a capture probe,

the optional B subunit, at each occurrence, independently comprises a hydrophilic moiety; and

the C subunit, at each occurrence, independently comprises a covalent attachment W to the outer surface of the substrate, wherein W has one of the following structures:

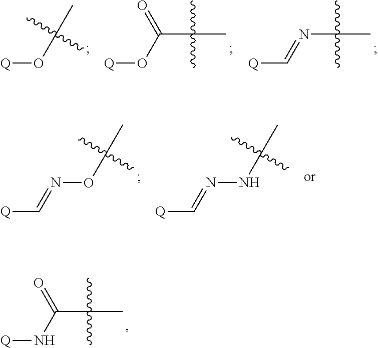

wherein Q is the outer surface of the substrate, and wherein the reactivity of the first and second thermochemically reactive groups are orthogonal to each other.

The present application also provides methods for preparing the disclosed solid substrates. For example, in one embodiment the method comprises:

A) providing a solid substrate comprising a plurality of hydroxyl, carbonyl or amine functional groups, or combinations thereof covalently bound to the outer surface thereof, wherein the hydroxyl and carbonyl functional groups are bound directly to the solid substrate without intervening linkers, and the amine functional groups are bound to the solid substrate through a linker comprising an imine bond, the imine bond being bound directly to the solid substrate without an intervening linker; and

B) contacting a polymer comprising D and optional E and F subunits with the solid substrate under conditions sufficient to form a covalent bond between at least one of the hydroxyl, carbonyl or amine functional groups and the D subunit, wherein:

the D subunit, at each occurrence, independently comprises a first reactive group, wherein the first reactive group is a thermochemically reactive group capable of forming a covalent bond with an alcohol, carbonyl or amine functional group on a solid substrate or capture probe;

the E subunit, at each occurrence, independently comprises a hydrophilic moiety; and

the F subunit, at each occurrence, independently comprises a second reactive group, wherein the second reactive group is a cycloaddition or conjugate addition reactive group having a reactivity specific for covalent bond formation with a target functional group on a capture probe via a cycloaddition or 1,4-conjugate addition reaction,

wherein the reactivity of the first reactive group and the second reactive group are orthogonal to each other.

Still other embodiments provide a method for determining the presence or absence of a target analyte molecule, the method comprises:

a) providing a solid support as described herein, wherein the A subunit comprises a capture probe covalently bound thereto;

b) contacting an analyte probe with the solid support; and

c) detecting the presence or absence of a signal produced from interaction of the capture probe with the analyte probe.

Polymers and functionalized solid substrates for preparation of the solid supports are also provided. For example, in one embodiment the present disclosure provides a solid support comprising a plurality of primary amine functional groups covalently bound to an outer surface of the solid substrate, wherein the amine functional groups are bound to the solid substrate through a linker comprising an imine bond.

In other embodiments, the disclosure is directed to a polymer comprising G, H and optional I subunits, wherein:

the G subunit, at each occurrence, independently comprises:
a) a first thermochemically reactive group, wherein the first thermochemically reactive group is capable of forming a covalent bond with an alcohol, carbonyl or amine group;
b) a second thermochemically reactive group, wherein the second thermochemically reactive group is a cycloaddition or conjugate addition reactive group having a reactivity specific for covalent bond formation with a target functional group via a cycloaddition or 1,4-conjugate addition reaction;

the H subunit, at each occurrence, has the following structure:

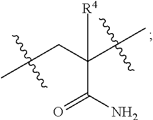

and

the optional I subunit, at each occurrence, independently comprises a hydrophilic moiety and has one of the following structures:

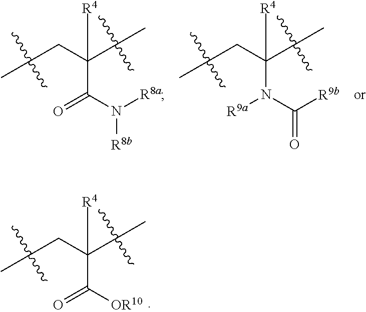

wherein:

R⁴ is at each occurrence, independently H or C₁-C₆ alkyl;

R^{8a} is H, C₁-C₆ alkyl or hydroxylalkyl;

R^{8b} is C₁-C₆ alkyl or hydroxylalkyl

R^{9a} and R^{9b} are each independently H, C₁-C₆ alkyl or hydroxylalkyl or R^{9a} and R^{9b}, together with the nitrogen atom to which they are bound, join to form a heterocyclic ring; and

R¹⁰ is hydroxylalkyl,

wherein the reactivity of the first and second thermochemically reactive groups are orthogonal to each other.

These and other aspects of the invention will be apparent upon reference to the following detailed description. To this end, various references are set forth herein which describe in more detail certain background information, procedures, compounds and/or compositions, and are each hereby incorporated by reference in their entirety.

### BRIEF DESCRIPTION OF THE DRAWINGS

In the figures, identical reference numbers identify similar elements. The sizes and relative positions of elements in the figures are not necessarily drawn to scale and some of these elements are arbitrarily enlarged and positioned to improve figure legibility. Further, the particular shapes of the elements as drawn are not intended to convey any information regarding the actual shape of the particular elements, and have been solely selected for ease of recognition in the figures.

FIGS. 1A, 1B, 1C, 1D, 1E and 1F depict exemplary embodiments of the solid support and preparation thereof.

FIGS. 2A, 2B and 2C illustrate exemplary analytical methods.

FIGS. 3A and 3B are ¹⁹F NMR spectra of exemplary polymers.

FIGS. 4A, 4B and 4C show results of a solid support subjected to multiple thermocycles.

FIG. 5 presents data for multiple water contact angle analyses of exemplary solid supports.

FIG. 6 is a bar graph showing the water contact angle of various solid supports before and after capping with different reagents and temperatures.

FIG. 7 is a graph illustrating the switchability of the water contact angle of various solid supports using different solvent systems. Polymer D=copoly(DMA-co-PFPA) containing 35.6 mol % of DMA.

### DETAILED DESCRIPTION OF THE INVENTION

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is as “including, but not limited to”.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

A “solid support” as used herein refers to a substrate which comprises a polymer and/or capture probe immobilized thereto. In some embodiments, the polymers are immobilized to the substrate via covalent bonds, with or without an intervening linker moiety which is immobilized to the substrate. The linker may be immobilized to the substrate through one or more covalent bonds or by other interactions, such as ionic interactions. Throughout the specification, certain embodiments refer to solid supports as devices.

“Substrate” or “solid substrate” refers to an object or substance used as a support or base for immobilizing the described polymers. Generally the substrate is a solid object and is not magnetic. The substrate can have any shape depending on the desired application, for example the substrate may be provided as a planar substrate, though the substrate can have any useful shape or configuration. Exemplary materials for substrates are provided herein below.

“Thermochemically reactive group” refers to a reactive group whose reactivity does not require UV or other sources of radiation for reactivity. Exemplary thermochemically reactive groups include, but are not limited to, activated esters (e.g., pentafluorophenyl ester, “PFP”), epoxides, azlactones, activated hydroxyls, maleimide and the like, as well as cycloaddition and conjugate addition reactive groups.

“Cycloaddition reactive group” refers to a thermochemically reactive group which is specific for formation of a cyclic moiety upon reaction with a complementary functional group. Exemplary cycloaddition reactive groups include, but are not limited to, alkynes and azides which form a triazole moiety via a cycloaddition reaction. Other examples include dienes and dienophiles which react via a Diels-Alder type cycloaddition with the appropriate complementary functional group.

“Conjugate addition reactive group” refers to a thermochemically reactive group which is specific for reaction in a conjugate addition reaction. For example, compounds containing α,β unsaturated carbonyl groups and nucleophiles capable of reacting with the same in a 1,4-conjugate addition reaction are examples of conjugate addition reactive groups.

The “outer surface” or “surface” of a substrate refers to the outermost portion substrate. In some instances the outer surface will be the outer surface of the native substrate. In other examples, the substrate will comprise a first surface which is the outer surface of the native substrate, and immobilized thereto is linker or a “tie layer” which is referred to as a second surface. Polymers immobilized (covalently or through other means) to the “outer surface” or to the “surface” of a substrate includes immobilization of the polymer to either the native substrate surface or to the second surface (linker or tie layer, etc.) or combinations thereof. The outer surface can be (1) the native surface of the substrate, (2) the first surface derived from plasma treatment, or (3) the second surface having linkers or a ‘tie-layer.’

“Immobilizing” or “immobilized” with respect to a support includes covalent conjugation, non-specific association, ionic interactions and other means of adhering a substance (e.g., polymer) to a substrate.

A “polymer” refers to a molecule having one or more repeating subunits. The subunits (“monomers”) may be the same or different and may occur in any position or order within the polymer. Polymers may be of natural or synthetic origin. The present invention includes various types of polymers, including polymers having ordered repeating subunits, random co-polymers and block co-polymers. Polymers having two different monomer types are referred to as co-polymers, and polymers having three different types of monomers are referred to as terpolymers, and so on.

A “random polymer” refers to a polymer wherein the subunits are connected in random order along a polymer chain. Random polymers may comprise any number of different subunits. In certain embodiments, the polymers described herein are “random co-polymers” or “random co-terpolymers”, meaning that the polymers comprise two or three different subunits, respectively, connected in random order. The individual subunits may be present in any molar ratio in the random polymer, for example each subunit may be present in from about 0.1 molar percent to about 99.8 molar percent, relative to moles of other subunits in the polymer. In some embodiments, the subunits of a random co-polymer may be represented by the following general structure:

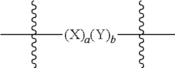

wherein X and Y are independently unique monomer subunits, and a and b are integers representing the number of each subunit within the polymer. For ease of illustration, the above structure depicts a linear connectivity of X and Y; however, it is to be emphasized that random co-polymers (e.g., random co-polymers, random co-terpolymers and the like) of the present invention are not limited to polymers having the depicted connectivity of subunits, and the subunits in a random polymer can be connected in any random sequence, and the polymers can be branched. Thus, structures of polymers depicted herein, for example structure (I), are meant to include polymers having subunits connected in any order.

A “block co-polymer” refers to a polymer comprising blocks of different subunits or different blocks of polymerized monomers.

A “functional group” is a portion of a molecule having a specific type of reactivity (e.g., acidic, basic, nucleophilic, electrophilic, etc). “Reactive groups” are a type of functional group. Non-limiting examples of functional groups include azides, alkynes, amine, alcohols and the like. A “target functional group” is any functional group with which another functional group is intended to react. A “hydrophilic functional group” is a functional group having hydrophilic properties. A hydrophilic functional group generally tends to increase the overall molecule's solubility in polar solvents such as water.

“Covalent conjugation” refers to formation of a covalent bond by reaction of two or more functional groups.

“Orthogonal” or “orthogonal reactivity” refers to reactivity properties of functional groups and/or reactive groups. If two reactive groups have orthogonal reactivity it is meant that one of the reactive groups will react with a target functional group under conditions in which the second reactive group does not react to a substantial extent with the target functional group, and vice versa.

“Initiator” is a molecule used to initiate a polymerization reaction. Initiators for use in preparation of the disclosed polymers are well known in the art. Representative initiators include, but are not limited to, initiators useful in atom transfer radical polymerization, living polymerization, the AIBN family of initiators and benzophenone initiators. An “initiator residue” is that portion of an initiator which becomes attached to a polymer through radical or other mechanisms. In some embodiments, initiator residues are attached to the terminal end(s) of the disclosed polymers.

“Click chemistry” refers to reactions that have at least the following characteristics: (1) exhibits functional group orthogonality (i.e., the functional portion reacts only with a reactive site that is complementary to the functional portion, without reacting with other reactive sites); and (2) the resulting bond is irreversible (i.e., once the reactants have been reacted to form products, decomposition of the products into reactants is difficult). Optionally, “click” chemistry can further have one or more of the following characteristics: (1) stereospecificity; (2) reaction conditions that do not involve stringent purification, atmospheric control, and the like; (3) readily available starting materials and reagents; (4) ability to utilize benign or no solvent; (5) product isolation by crystallization or distillation; (6) physiological stability; (7) large thermodynamic driving force (e.g., 10-20 kcal/mol); (8) a single reaction product; (9) high (e.g., greater than 50%) chemical yield; and (10) substantially no byproducts or byproducts that are environmentally benign byproducts.

Examples of reactions using “click” functionalities can include, but are not limited to, addition reactions, cycloaddition reactions, nucleophilic substitutions, and the like. Examples of cycloaddition reactions can include Huisgen 1,3-dipolar cycloaddition, Cu(I) catalyzed azide-alkyne cycloaddition, and Diels-Alder reactions. Examples of addition reactions include addition reactions to carbon-carbon double bonds such as epoxidation and dihydroxylation. Nucleophilic substitution examples can include nucleophilic substitution to strained rings such as epoxy and aziridine compounds. Other examples can include formation of ureas and amides. Some additional description of click chemistry can be found in Huisgen, Angew. Chem. Int. Ed., Vol. 2, No. 11, 1963, pp. 633-696; Lewis et al., Angew. Chem. Int. Ed., Vol. 41, No. 6, 2002, pp. 1053-1057; Rodionov et al., Angew. Chem. Int. Ed., Vol. 44, 2005, pp. 2210-2215; Punna et al., Angew. Chem. Int. Ed., Vol. 44, 2005, pp. 2215-2220; Li et al., J. Am. Chem. Soc., Vol. 127, 2005, pp. 14518-14524; Himo et al., J. Am. Chem. Soc., Vol. 127, 2005, pp. 210-216; Noodleman et al., Chem. Rev., Vol. 104, 2004, pp. 459-508; Sun et al., Bioconjugate Chem., Vol. 17, 2006, pp. 52-57; and Fleming et al., Chem. Mater., Vol. 18, 2006, pp. 2327-2334, the contents of which are hereby incorporated by reference herein in their entireties.

“Click reactivity” refers to a functional group capable of reacting under click chemistry conditions.

A “click functional group” is a functional group which results from reaction of two functional groups having click reactivity, for example a triazole moiety and the like.

A reactive group having “reactivity specific for” a target functional group means the reactive group will react preferentially with the target functional group under the reaction conditions and side reactions with other functional groups are minimized or absent. Similarly, a reactive group having reactivity specific for conjugation with a capture probe means the reactive group will conjugate preferentially with the capture probe under the reaction conditions and side reactions with other functional groups are minimized or absent.

“Analyte” or “analyte molecule” refers to a compound or molecule which is the subject of an analysis, for example an analyte molecule may be of unknown structure and the analysis includes identification of the structure. Analyte molecules include any number of common molecules, including DNA, proteins, peptides and carbohydrates, organic and inorganic molecules, metals (including radioactive isotopes), and the like. Analytes include viruses, bacteria, plasmodium, fungi, as well as metals and bio-warfare, bio-hazard and chemical warfare materials. Analytes also include analyte probes as defined herein.

A “capture probe” is a molecule capable of interacting with an analyte molecule, for example by hydrogen bonding (e.g., DNA hybridization), sequestering, covalent bonding, ionic interactions, and the like. Exemplary capture probes include oligonucleotides which are capable of sequence specific binding (hybridization) with oligonucleotide probes or flaps, oligosaccharides (e.g. lectins) and proteins. In some embodiments capture probes comprise a fluorophore label. For example the capture probe may comprise a fluorophore label and an analyte molecule (e.g., analyte probe) may comprise a quencher, and the presence of the analyte molecule is detected by an absence of a fluorescent signal from the capture probe (since the fluorescence is quenched upon interaction with the quencher). In related embodiments, the capture probe comprises a quencher. In these embodiments, the fluorescence of a fluorescently labeled analyte molecule is quenched upon capture by the capture probe.

“Probe” or “analyte probe” refers to a molecule used for indirect identification of an analyte molecule. For example, a probe may carry sequence information which uniquely identifies an analyte molecule. Exemplary probes include oligonucleotides and the like.

“Flap” refers to an optional portion of a probe. In certain embodiments a flap contains sequence information to uniquely identify the probe (and thus the analyte molecule). A flap may be cleaved from the remainder of the probe (for example under PCR conditions) and hybridize with a capture probe on a solid support. The presence of the bound flap on the solid support indicates the presence of a particular analyte.

“Amino” refers to the —NH₂ radical.

“Azide” refers to the —N₃ radical.

“Aziridine” refers to a three-membered, nitrogen containing ring.

“Cyano” or “nitrile” refers to the —CN radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thiirane” refers to a three-membered, sulfur containing ring.

“Thiol” refers to the —SH substituent.

“Thioxo” refers to the ═S substituent.

“Sulfo” refers to the —SO₃M substituent, wherein M is H or a cation such as K, Na, or ammonium (i.e., N⁺(R^{a}R^{b}R^{c}R^{d}) where R^{a}, R^{b}, R^{c} and R^{d} is independently H or C₁-C₆ alkyl).

“Alkyl” refers to a straight or branched hydrocarbon chain radical consisting solely of carbon and hydrogen atoms, which is saturated or unsaturated (i.e., contains one or more double (i.e., alkene) and/or triple bonds (i.e., alkyne)), having from one to twelve carbon atoms (C₁-C₁₂ alkyl), preferably one to eight carbon atoms (C₁-C₈ alkyl) or one to six carbon atoms (C₁-C₆ alkyl), and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylhexyl, 2-methylhexyl, ethenyl, prop-1-enyl, but-1-enyl, pent-1-enyl, penta-1,4-dienyl, ethynyl, propynyl, butynyl, pentynyl, hexynyl, and the like. Unless stated otherwise specifically in the specification, an alkyl group may be optionally substituted.

“Alkylene” or “alkylene chain” refers to a straight or branched divalent hydrocarbon chain linking the rest of the molecule to a radical group, consisting solely of carbon and hydrogen, which is saturated or unsaturated (i.e., contains one or more double and/or triple bonds), and having from one to twelve carbon atoms, e.g., methylene, ethylene, propylene, n-butylene, ethenylene, propenylene, n-butenylene, propynylene, n-butynylene, and the like. The alkylene chain is attached to the rest of the molecule through a single or double bond and to the radical group through a single or double bond. The points of attachment of the alkylene chain to the rest of the molecule and to the radical group can be through one carbon or any two carbons within the chain. Unless stated otherwise specifically in the specification, an alkylene chain may be optionally substituted.

“Alkoxy” refers to a radical of the formula —ORₐ where Rₐ is an alkyl radical as defined above containing one to twelve carbon atoms. Unless stated otherwise specifically in the specification, an alkoxy group may be optionally substituted.

“Alkylamino” refers to a radical of the formula —NHRₐ or —NRₐRₐ where each Rₐ is, independently, an alkyl radical as defined above containing one to twelve carbon atoms. Unless stated otherwise specifically in the specification, an alkylamino group may be optionally substituted.

“Alkyloxycarbonyl” refers to a radical of the formula —CO(═O)Rₐ where Rₐ is an alkyl radical as defined. “Hydroxylalkyloxycarbonyl” is an alkyloxycarbonyl comprising at least one hydroxyl substitutent. Unless stated otherwise specifically in the specification, an alkyloxycarbonyl and hydroxylalkyloxycarbonyl groups may be optionally substituted as described below.

“Thioalkyl” refers to a radical of the formula —SRₐ where Rₐ is an alkyl radical as defined above containing one to twelve carbon atoms. Unless stated otherwise specifically in the specification, a thioalkyl group may be optionally substituted.

“Aryl” refers to a hydrocarbon ring system radical comprising hydrogen, 6 to 18 carbon atoms and at least one aromatic ring. For purposes of this invention, the aryl radical may be a monocyclic, bicyclic, tricyclic or tetracyclic ring system, which may include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” or the prefix “ar-” (such as in “aralkyl”) is meant to include aryl radicals that are optionally substituted.

“Aralkyl” refers to a radical of the formula —R_{b}—R_{c} where R_{b} is an alkylene chain as defined above and R_{c} is one or more aryl radicals as defined above, for example, benzyl, diphenylmethyl and the like. Unless stated otherwise specifically in the specification, an aralkyl group may be optionally substituted.

“Cycloalkyl” or “carbocyclic ring” refers to a stable non-aromatic monocyclic or polycyclic hydrocarbon radical consisting solely of carbon and hydrogen atoms, which may include fused or bridged ring systems, having from three to fifteen carbon atoms, preferably having from three to ten carbon atoms, and which is saturated or unsaturated and attached to the rest of the molecule by a single bond. Monocyclic radicals include, for example, cyclopropyl, cyclobutyl, cyclopentyl, cyclohexyl, cycloheptyl, and cyclooctyl. Polycyclic radicals include, for example, adamantyl, norbornyl, decalinyl, 7,7-dimethyl-bicyclo[2.2.1]heptanyl, and the like. Unless otherwise stated specifically in the specification, a cycloalkyl group may be optionally substituted.

“Cycloalkylalkyl” refers to a radical of the formula —R_{b}R_{d} where R_{b} is an alkylene chain as defined above and R_{d} is a cycloalkyl radical as defined above. Unless stated otherwise specifically in the specification, a cycloalkylalkyl group may be optionally substituted.

“Fused” refers to any ring structure described herein which is fused to an existing ring structure in the compounds of the invention. When the fused ring is a heterocyclyl ring or a heteroaryl ring, any carbon atom on the existing ring structure which becomes part of the fused heterocyclyl ring or the fused heteroaryl ring may be replaced with a nitrogen atom.

“Halo” or “halogen” refers to bromo, chloro, fluoro or iodo.

“Haloalkyl” refers to an alkyl radical, as defined above, that is substituted by one or more halo radicals, as defined above, e.g., trihalomethyl, such as trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group may be optionally substituted.

“Heterocyclyl” or “heterocyclic ring” refers to a stable 3- to 18-membered non-aromatic ring radical which consists of two to twelve carbon atoms and from one to six heteroatoms selected from the group consisting of nitrogen, oxygen and sulfur. Unless stated otherwise specifically in the specification, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the heterocyclyl radical may be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, Unless stated otherwise specifically in the specification, a heterocyclyl group may be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group may be optionally substituted.

“Heterocyclylalkyl” refers to a radical of the formula —R_{b}Rₑ where R_{b} is an alkylene chain as defined above and Rₑ is a heterocyclyl radical as defined above, and if the heterocyclyl is a nitrogen-containing heterocyclyl, the heterocyclyl may be attached to the alkyl radical at the nitrogen atom. Unless stated otherwise specifically in the specification, a heterocyclylalkyl group may be optionally substituted.

“Heteroaryl” refers to a 5- to 14-membered ring system radical comprising hydrogen atoms, one to thirteen carbon atoms, one to six heteroatoms selected from the group consisting of nitrogen, oxygen and sulfur, and at least one aromatic ring. For purposes of this invention, the heteroaryl radical may be a monocyclic, bicyclic, tricyclic or tetracyclic ring system, which may include fused or bridged ring systems; and the nitrogen, carbon or sulfur atoms in the heteroaryl radical may be optionally oxidized; the nitrogen atom may be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group may be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group may be optionally substituted.

“Heteroarylalkyl” refers to a radical of the formula —R_{b}R_{f} where R_{b} is an alkylene chain as defined above and R_{f} is a heteroaryl radical as defined above. Unless stated otherwise specifically in the specification, a heteroarylalkyl group may be optionally substituted.

“Hydroxylalkyl” is an alkyl, as defined above, comprising one or more hydroxyl substituents. Unless specifically stated otherwise, a hydroxylalkyl may be optionally substituted.

The term “substituted” as used herein means any of the above groups (i.e., alkyl, alkylene, alkoxy, alkyloxycarbonyl alkylamino, thioalkyl, aryl, aralkyl, cycloalkyl, cycloalkylalkyl, haloalkyl, heterocyclyl, N-heterocyclyl, heterocyclylalkyl, heteroaryl, hydroxylalky, hydroxylalkyloxycarbonyl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}CO(═O)Rₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, N-heteroaryl and/or heteroarylalkyl group. In addition, each of the foregoing substituents may also be optionally substituted with one or more of the above substituents.

“Stable compound” and “stable structure” are meant to indicate a compound that is sufficiently robust to survive isolation to a useful degree of purity from a reaction mixture.

“Optional” or “optionally” means that the subsequently described event of circumstances may or may not occur, and that the description includes instances where said event or circumstance occurs and instances in which it does not. For example, “optionally substituted aryl” means that the aryl radical may or may not be substituted and that the description includes both substituted aryl radicals and aryl radicals having no substitution.

Often crystallizations or precipitations produce a solvate of the compound of the invention. As used herein, the term “solvate” refers to an aggregate that comprises one or more molecules of a compound of the invention with one or more molecules of solvent. The solvent may be water, in which case the solvate may be a hydrate. Alternatively, the solvent may be an organic solvent. Thus, the compounds of the present invention may exist as a hydrate, including a monohydrate, dihydrate, hemihydrate, sesquihydrate, trihydrate, tetrahydrate and the like, as well as the corresponding solvated forms. The compound of the invention may be true solvates, while in other cases, the compound of the invention may merely retain adventitious water or be a mixture of water plus some adventitious solvent.

The compounds of the invention, or their salts or tautomers may contain one or more asymmetric centers and may thus give rise to enantiomers, diastereomers, and other stereoisomeric forms that may be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another.

A “tautomer” refers to a proton shift from one atom of a molecule to another atom of the same molecule. The present invention includes tautomers of any said compounds.

### A. Solid Supports

As noted above, one aspect of the present disclosure is directed to solid supports comprising a plurality of polymers covalently bound to a solid substrate. The polymers generally comprise reactive functional groups for immobilization (e.g., covalent conjugation) of biomolecules, such as DNA, or other analytes. The solid supports provide numerous advantages over previously described solid supports, such as facile assembly without the need for a tie layer to immobilize the polymer to the solid substrate. Favorable water contact angles are also realized via the presently described solid supports. Accordingly, the solid supports find particular utility in high resolution/high density array analyses of various analytes, such as DNA.

PCR microarrays on plastic substrates require high Tg greater than 120° C., low water absorption less than 1%, greater than 90% optical transparency over the range of 400-800 nm, and low fluorescent background. A few commercially available polymers having the above characteristics tend to be chemically inert. Wet-chemical surface modification of this type of polymers is tedious and/or cost prohibiting. Often the substrate polymer is unstable to common processing solvents. The present inventors have discovered that oxygen plasma treatment to hydroxylate the substrate surface for polymer immobilization is a simple, low-cost, and effective approach.

An exemplary solid support comprising a rigid thermoplastic monolith may be chemically activated directly by atmospheric pressure oxygen plasma, or by other plasma methods, to generate hydroxyl (or other oxygenated) groups on the surface. Other substrate surface plasma treatments are also contemplated, including ammonia plasma treatment, nitrogen plasma treatment and nitrogen/hydrogen plasma in ratios from between about 1:3 to about 10:1 to generate amino groups on the surface. Plasma treatment provides a convenient, rapid, automatable, and reproducible technique for surface functionalization compared to methods that rely on adhesion of a preliminary layer for subsequent immobilization of a functional layer.

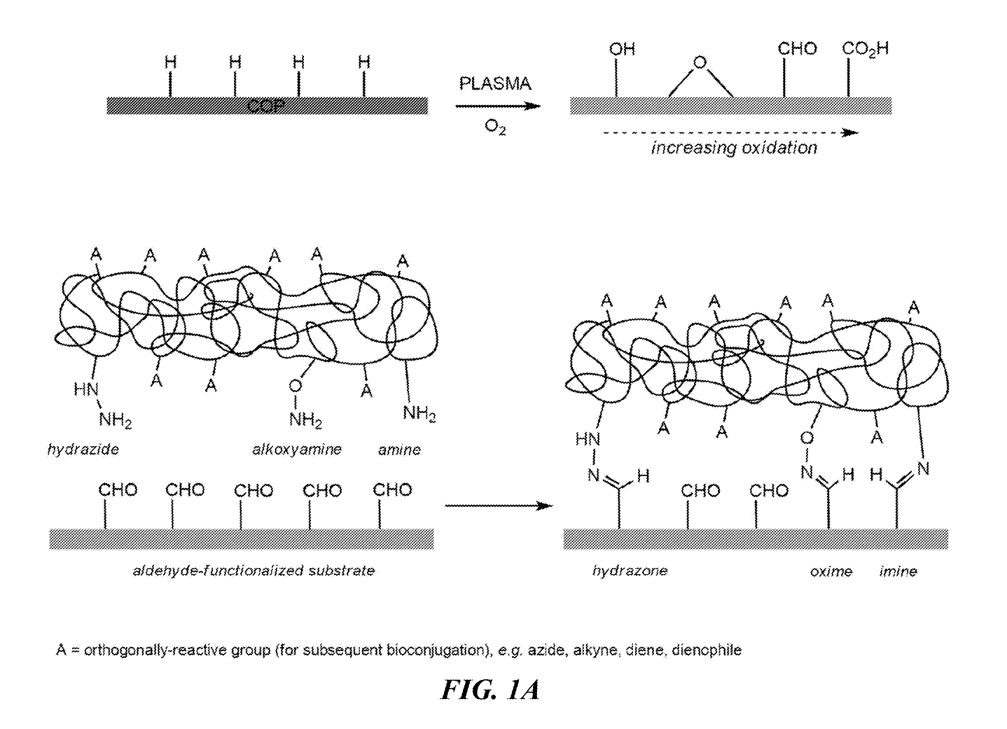

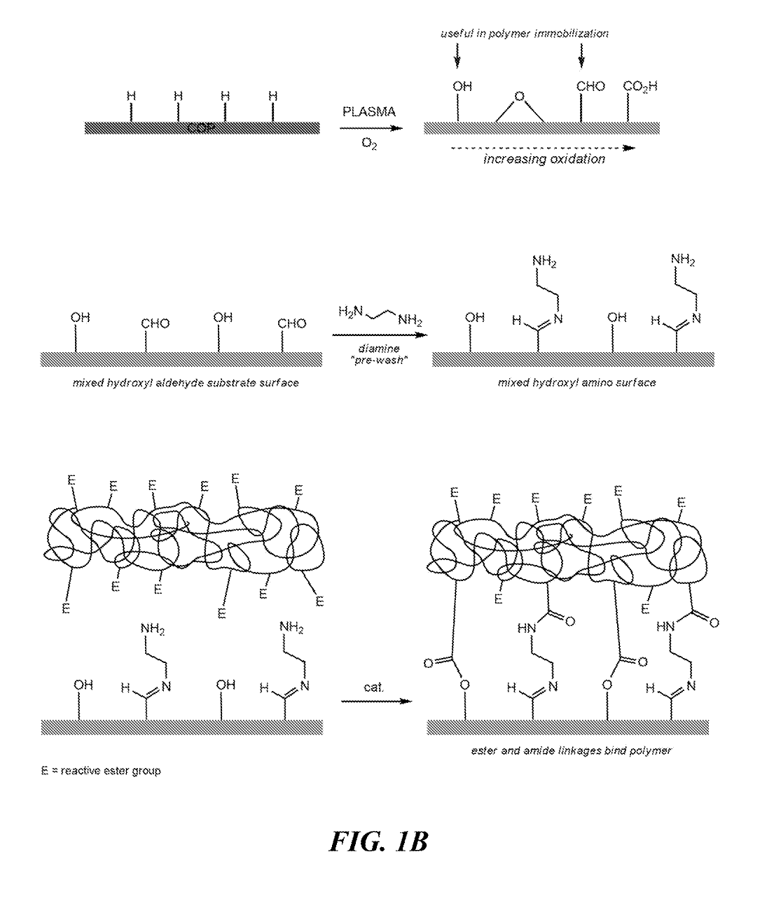

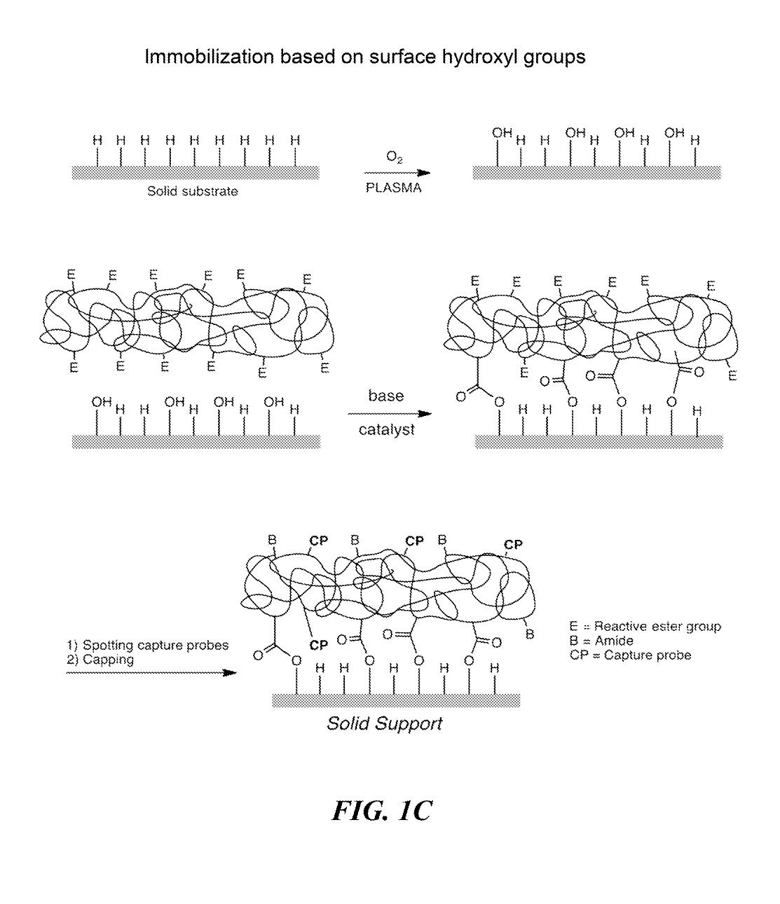

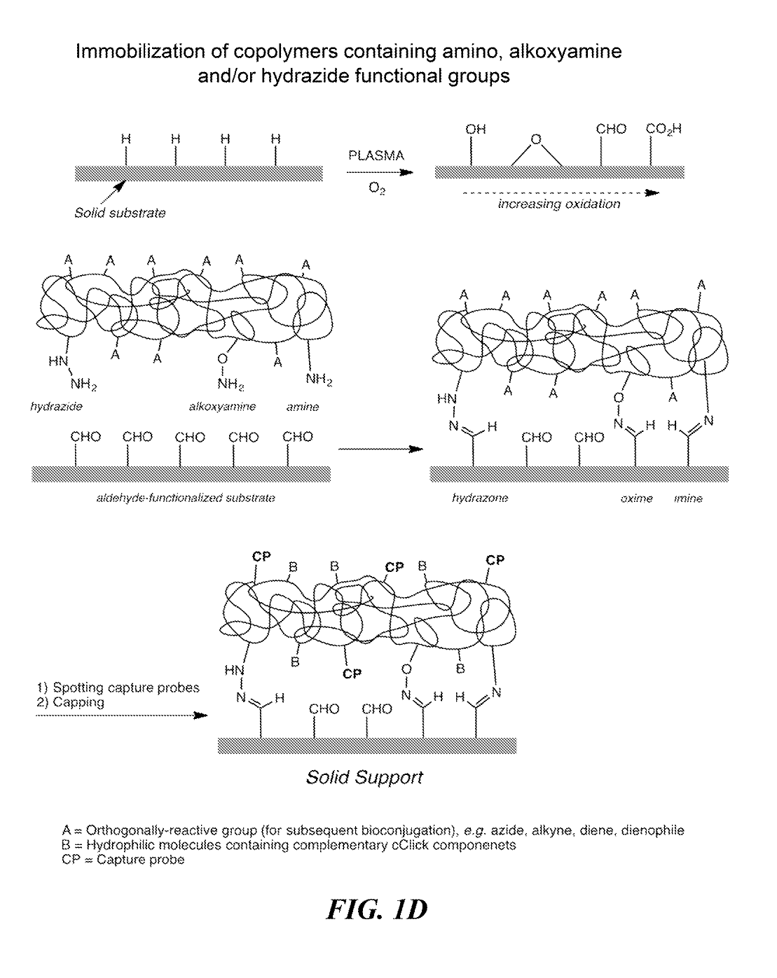

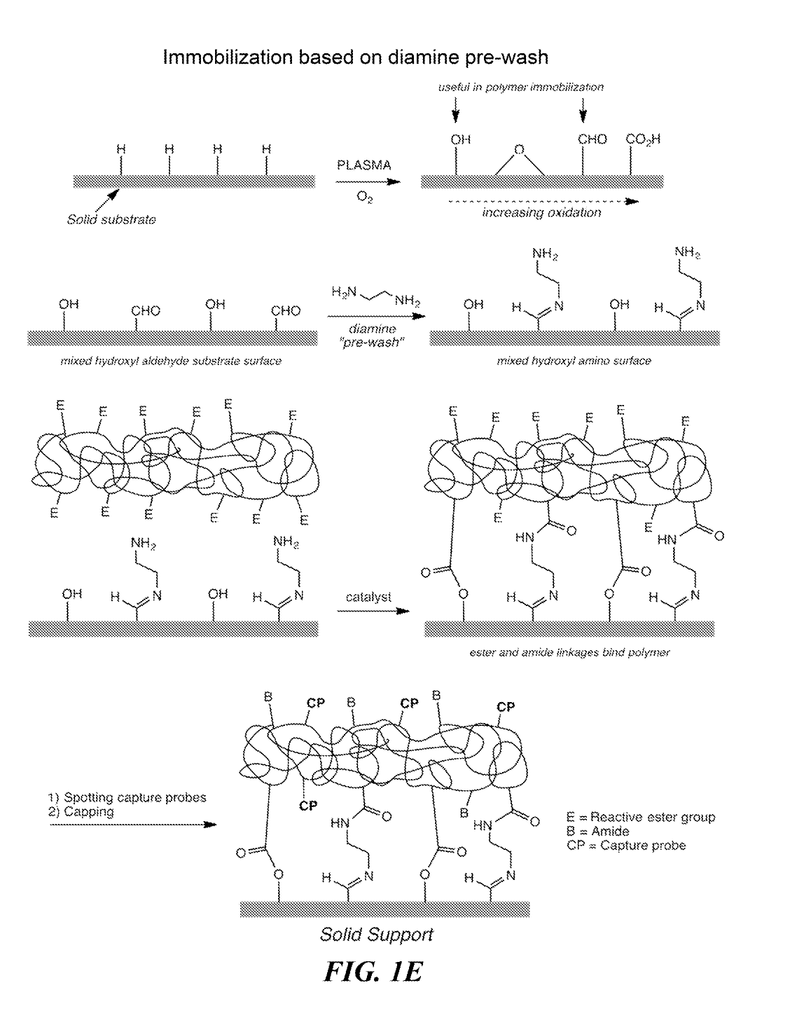

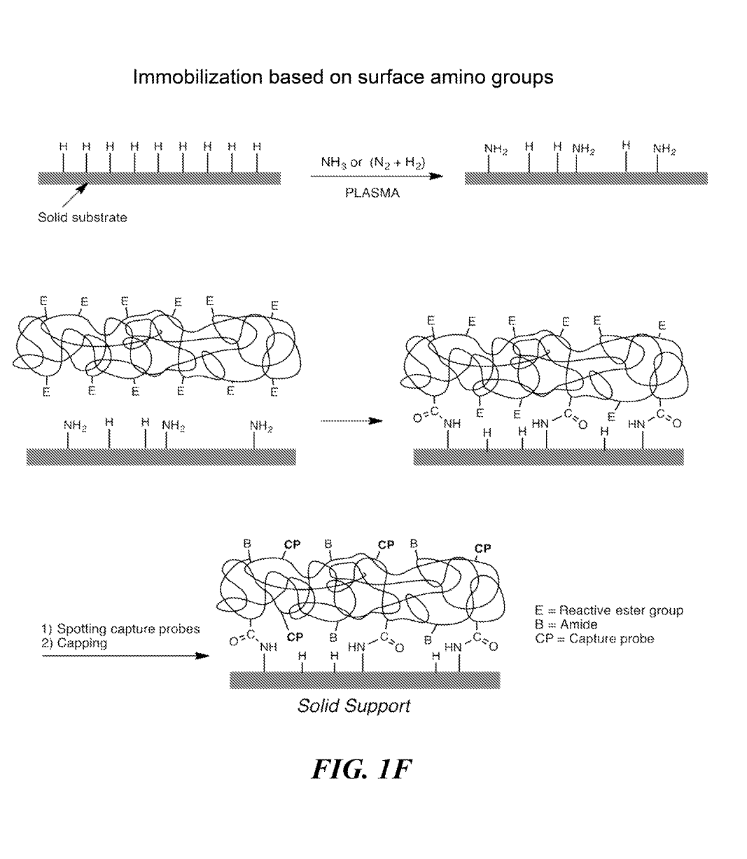

The solid supports of the present disclosure can be better understood in reference to FIGS. 1A-1F. As seen in FIG. 1A, a solid substrate can be provided with various oxidized functional groups, including hydroxyl, epoxide, aldehyde and carboxy groups, by plasma treatment of an appropriate substrate. Substrates useful in this regard are described in more detail below. The solid supports are then prepared by reaction of a polymer comprising an appropriate reactive group. For example, FIG. 1A depicts reaction of a polymer comprising hydrazide, alkoxyamine and amine reactive groups with a surface bound aldehyde to form hydrazone, oxime and imine covalent bonds, respectively. Advantageously, the polymers are thereby covalently bound directly to the surface of the solid substrate without an intervening linker or “tie layer.” For ease of illustration, FIG. 1A depicts multiple reactive functional groups in the same polymer; however, it is to be understood that the invention includes various embodiments wherein the polymer comprises a single type of functional group.

FIG. 1B depicts another embodiment of the solid supports. Again, a solid substrate is treated with atmospheric pressure O₂ plasma (APOP) to obtain various oxidized functional groups on the surface of the substrate. The substrate is then washed with a diamine (e.g., ethylene diamine) to incorporate free amine moieties bound to the substrate via imine bonds. A polymer comprising appropriate reactive groups, such as activated esters, can then be covalently bound to the solid substrate by reaction with alcohols on the substrate surface (to form a new ester) and/or reaction with an amine (to form an amide). While FIG. 1A depicts a solid support comprising both amide and ester bonds to the polymer, one of ordinary skill in the art will understand that certain embodiments include substrates having either ester or amide bonds. For example, without the diamine treatment, the solid support will primarily comprise ester bond when the polymer comprises activated ester. Conversely, conditions employed during the diamine wash can be controlled such that the substrate surface primarily comprises amines and the polymer will primarily be bound to the substrate via amide bonds (when the polymer comprises activated esters).

Accordingly, in one embodiment the solid support comprises:

a substrate having an outer surface; and

a plurality of polymers covalently bound to the outer surface of the substrate, the polymers each comprising at least one A and C subunit and optionally comprising one or more B subunits, wherein:

the A subunit, at each occurrence, independently comprises:
a) a first thermochemically reactive group, wherein the first thermochemically reactive group is capable of forming a covalent bond with an alcohol, carbonyl or amine group on a capture probe;
b) a second thermochemically reactive group, wherein the second thermochemically reactive group is cycloaddition or conjugate addition reactive group having a reactivity specific for covalent bond formation with a target functional group on a capture probe via a cycloaddition or 1,4-conjugate addition reaction; or
c) a covalent bond to a capture probe,

the optional B subunit, at each occurrence, independently comprises a hydrophilic moiety; and

the C subunit, at each occurrence, independently comprises a covalent attachment W to the outer surface of the substrate, wherein W has one of the following structures:

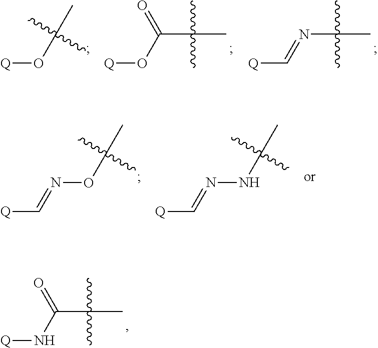

wherein Q is the outer surface of the substrate, and wherein the reactivity of the first and second thermochemically reactive groups are orthogonal to each other.

In some embodiments, W has one of the following structures:

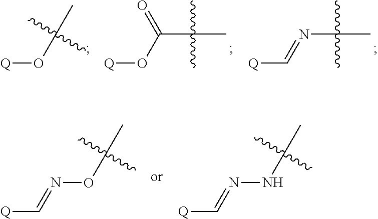

In certain other embodiments, the polymers have the following formula (I):

T¹-(A)ₓ(B)_{y}(C)_{z}-T² (I)

wherein:

A, B and C represent the A, B and C subunits, respectively;

T¹ and T² are each independently absent or polymer terminal groups selected from H, alkyl and an initiator residue;

x and z are independently an integer from 1 to 50,000; and

y is an integer from 0 to 50,000.

For example, in some embodiments the solid support has the following formula (II):

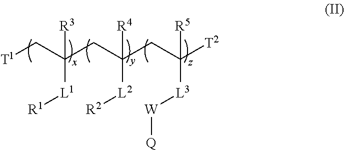

wherein:

R¹ is, at each occurrence, independently the first thermochemically reactive group, the second thermochemically reactive group or the covalent bond to the capture probe;

R² is, at each occurrence, independently the hydrophilic moiety;

W is, at each occurrence, independently the covalent attachment to the outer surface of the substrate;

Q is the outer surface of the substrate;

R³, R⁴ and R⁵ are, at each occurrence, independently H or C₁-C₆ alkyl;

L¹, L² and L³ are, at each occurrence, independently a direct bond or a linker up to 100 atoms in length;

T¹ and T² are each independently absent or polymer terminal groups selected from H, alkyl and an initiator residue;

x and z are each independently an integer from 1 to 50,000; and

y is an integer from 0 to 50,000.

In any one of the foregoing embodiments, at least one A subunit comprises a first thermochemically reactive group. In some embodiments the first thermochemically reactive group is an activated ester, for example in some aspects the first thermochemically reactive group has, at each occurrence, independently the following formula:

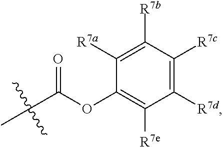

wherein R^{7a}, R^{7b}, R^{7c}, R^{7d} and R^{7e} are each independently H, halo, trihalomethyl, sulfo (i.e., —SO₃H and/or salts thereof), —CN, C₁-C₆ alkyloxycarbonyl, C₁-C₆ hydroxylalkyloxycarbonyl, nitro or polyethylene glycol, wherein the polyethylene glycol is linked to the phenyl moiety via an oxygen (ether) or carboxyl (amide or ester) linkage. For example, in certain embodiments, at least one of R^{7a}, R^{7b}, R^{7c}, R^{7d} or R^{7e} may independently be —CO₂R, wherein R is alkyl, hydroxylalkyl or alkoxy(polyethoxy)ethyl. In certain embodiments the polyethylene glycol moiety comprises from 50 to 3,000 ethylene oxide subunits.

In various embodiments of the foregoing, halo is fluoro. For example, in some embodiments at least one of R^{7a}, R^{7b}, R^{7c}, R^{7d} and R^{7e} is fluoro. In other embodiments, each of R^{7a}, R^{7b}, R^{7c}, R^{7d} and R^{7e} are fluoro. In another embodiment, each of R^{7a}, R^{7b}, R^{7d} and R^{7e} are fluoro, and R^{7e} is sulfo. In certain other embodiments of the foregoing, the first thermochemically reactive group comprises a 4-sulfotetrafluorophenyl ester (i.e., wherein each of R^{7a}, R^{7b}, R^{7d} and R^{7e} are fluoro, and R^{7c} is sulfo.) Advantageously, polymers comprising these types of fluorinated reactive moieties can be analyzed by ¹⁹F and/or ¹H NMR techniques to accurately determine the ratio between reactive monomers and diluent monomers in a polymer. The molar feed ratio does not always accurately predict the mol % of the subunits incorporated into the final polymer; however, the presence of one or more F atoms in certain embodiments of the present polymers allows for accurate determination of the actual molar composition of the polymers. Methods for such determination are provided in the examples.

In certain other embodiments, one of R^{7a}, R^{7b}, R^{7c}, R^{7d} or R^{7e} is nitro. For example, in some embodiments, one of R^{7a}, R^{7b}, R^{7c}, R^{7d} or R^{7e} is nitro and the remaining substituents are H.

In other embodiments of any of the solid supports described herein, at least one A subunit comprises the second thermochemically reactive group.

In some embodiments, the alkyne, alkylsilyl-protected alkyne, azide, nitrile, thiol, alkene, maleimide, butadiene, cyclopentadiene, aziridine, thiirane, diene, dienophile or 1,4-unsaturated carbonyl functional group.

In other embodiments, the second thermochemically reactive group comprises a cycloaddition reactive group. For example, in some embodiments the cycloaddition reactive group comprises, at each occurrence, independently an alkyne or azide functional group. Exemplary cycloaddition reactive groups have, at each occurrence, independently one of the following formulas:

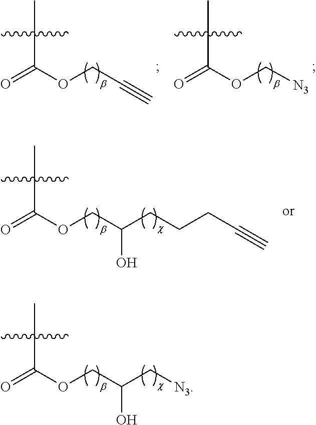

wherein β and χ are each independently integers ranging from 1 to 5.

In some examples, β is 1 or 3. In other examples, χ is 1.

In certain other embodiments, the cycloaddition reactive group has, at each occurrence, independently one of the following formulas:

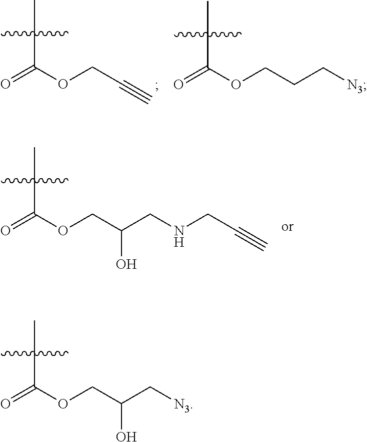

In other embodiments, the cycloaddition reactive group comprises, at each occurrence, independently a diene or dienophile functional group. For example, in some embodiments the cycloaddition reactive group comprises, at each occurrence, independently a α,β-unsaturated carbonyl, maleimidyl, acetylene dicarboxylic ester, cyclopentyldienyl, furanyl or N-alkylpyrrolyl moiety. Exemplary cycloaddition reactive groups in this regard have one of the following structures:

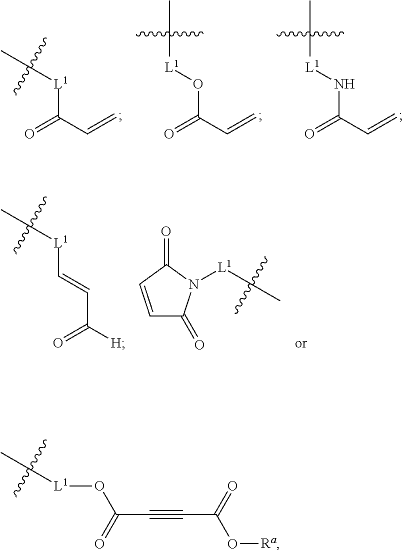

wherein R^{a} is C₁-C₆ alkyl and L¹ is a direct bond or a linker up to 100 atoms in length.

FIG. 1C depicts another embodiment of the solid supports. Again, a solid substrate is treated with atmospheric pressure O₂ plasma (APOP) to obtain hydroxyl functional groups on the outer surface. A polymer comprising appropriate reactive groups, such as activated esters, can then be covalently bound (immobilized) to the solid substrate to form a new ester. Catalysts (e.g., triethylamine) may be also be employed to increase reactivity of the activated esters.

FIG. 1D depicts another embodiment of the solid supports. As seen in FIG. 1D, a solid substrate can be provided with various oxidized functional groups, including hydroxyl, epoxide, aldehyde and carboxy groups, by oxygen plasma treatment of an appropriate substrate. The functionalized surface is then exposed to a polymer comprising appropriate reactive groups. For example, FIG. 1D depicts reaction of a polymer comprising hydrazide, alkoxyamine and amine reactive groups with a surface bound aldehyde to form hydrazone, oxime and imine covalent bonds, respectively. Advantageously, the polymers are thereby covalently bound directly to the surface of the solid substrate without an intervening linker or “tie layer.” For ease of illustration, FIG. 1D depicts multiple types of reactive functional groups in the same polymer; however, it is to be understood that the invention includes various embodiments wherein the polymer comprises a single type of functional group. The capture probes are then spotted to the functionalized polymer surface by bioconjugation to at least one of the orthogonally-reactive groups A, including but not limited to an azide, alkyne, diene, dienophile or reactive ester group. The solid support then undergoes ammonia capping, converting the remaining orthogonally reactive groups A into hydrophilic functional groups B, resulting in a hydrophilic surface having low water contact angles (e.g., less than 15 degrees) to reduce non-specific adsorption of biomolecules and air bubbles. In various embodiments, the reactive group A is selected from hydrazide, alkoxyamine and amine reactive groups.

FIG. 1E depicts another embodiment of the solid supports. Again, a solid substrate surface is treated with atmospheric pressure O₂ plasma (APOP) to obtain various oxidized functional groups, including hydroxyl, epoxide, aldehyde and carboxy groups. Illustrated is a mixture of hydroxyl and aldehyde substrates though other substrates, as would be known to one of skill in the art, are also envisioned. The functionalized surface is then subjected to a diamine pre-wash resulting in a mixed hydroxy amino surface. A polymer comprising appropriate reactive groups, such as activated esters, is then covalently bound to the functionalized solid substrate in the presence of an optional amine catalyst to form new ester and amide linkages to bind the polymer to the substrate surface. At least one reactive group in the polymer, which in certain embodiments is a copolymer comprising two types of subunits, reacts with at least one of the surface reactive groups. At least one of the remaining reactive groups in the polymer reacts with the capture probe in a subsequent spotting step to form a covalent amide bond. The solid support then undergoes ammonia capping to increase its surface hydrophilicity by replacing the ester groups on the polymer with amide groups.

FIG. 1F depicts another embodiment of the solid supports. A solid substrate surface is treated with atmospheric pressure NH₃ or (N₂+H₂) plasma to obtain amino functional groups. The solid supports are then prepared by reaction of a polymer comprising appropriate reactive groups. For example, FIG. 1F depicts reaction of a polymer comprising ester reactive groups with surface bound amino groups to form amide covalent bonds. At least one reactive group in the polymer, which in some embodiments is a copolymer comprising two types of subunits, reacts with at least one of the surface reactive groups. At least one of the remaining reactive groups in the polymer reacts with the capture probe in a subsequent spotting step to form a covalent amide bond. The solid support then undergoes ammonia capping to increase its surface hydrophilicity by replacing the ester groups on the polymer with amide groups. Advantageously, the polymers are thereby covalently bound directly to the surface of the solid substrate without an intervening linker or “tie layer.” For ease of illustration, the capture probes are then spotted and covalently bound to the functionalized polymer surface by interaction with the ester group. The solid support then undergoes ammonia capping to increase its surface hydrophilicity by replacing the ester groups on the polymer with amide groups.

In certain embodiments, the solid supports further comprise a capture probe immobilized thereto. For example, in some embodiments at least one A subunit comprises a covalent bond to the capture probe. The covalent bond is generally formed between one of the first or second thermochemically reactive groups and an appropriate reactive group on the capture probe. For example, when the first thermochemically reactive group is an ester, the covalent bond formed between the capture probe and the polymer can be an ester or amide (from reaction of an alcohol or amine on the capture probe). In certain embodiments, the covalent bond is an amidyl or amine bond to the capture probe. In certain embodiments, the covalent bond is an amidyl or thioester bond to the capture probe.

In other embodiments, the covalent bond between the polymer and the capture probe (when present) is formed between a cycloaddition reactive group on the polymer and a complementary reactive group on the capture probe. “Click” chemistry can be particularly useful in this regard. Accordingly, in some embodiments the cycloaddition reactive group is an alkyne or azide. In other embodiments, the covalent bond to the capture probe comprises a triazole moiety.

In other related embodiments, at least one A subunit has one of the following structures:

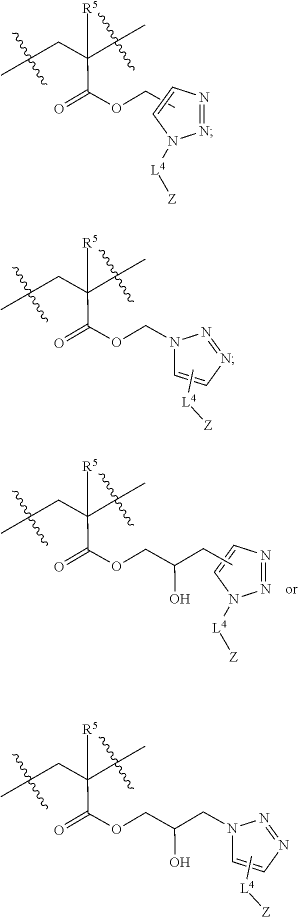

wherein:

R⁵ is H or C₁-C₆ alkyl;

L⁴ is an optional linker; and

Z is the capture probe or fragment thereof

The solid supports find utility for analysis of any number of analytes. In this regard the identity of the capture probe is not particularly limited and one of ordinary skill in the art will be able to envision the various capture probes useful in the context of the present solid supports. Although not limited, certain embodiments are directed to capture probes selected from a peptide, protein, glycosylated protein, glycoconjugate, aptomer, carbohydrate, polynucleotide, oligonucleotide and polypeptide. In certain embodiments, the capture probe is a polynucleotide. In other embodiments, the capture probe is DNA.

As noted above, the present solid supports advantageously comprise polymers covalently bound to the surface of a solid substrate. Accordingly, methods for preparation of the solid substrates are more commercially feasible and the resulting solid supports have many functional advantages over previously described solid supports, including advantageous WCA switching properties as described above. In certain embodiments, the covalent attachment (“W”) between the polymer and the solid substrate has, at each occurrence, independently one of the following structures:

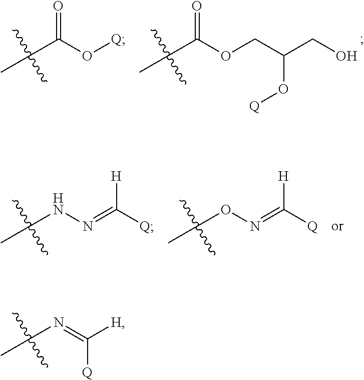

wherein Q is the solid substrate.

In some embodiments, W is

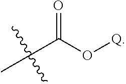

In other embodiments, W is

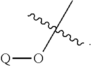

In other embodiments, W is

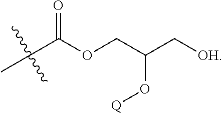

In more embodiments, W is

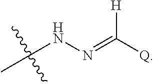

In yet other embodiments,

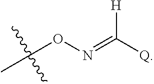

In other embodiments, W is

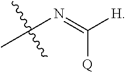

In still other embodiments, W is

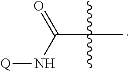

Solid supports comprising any combination of the foregoing W structures are also included within the scope of different embodiments of the invention.

In other embodiments, the C subunit has, at each occurrence, independently one of the following structures:

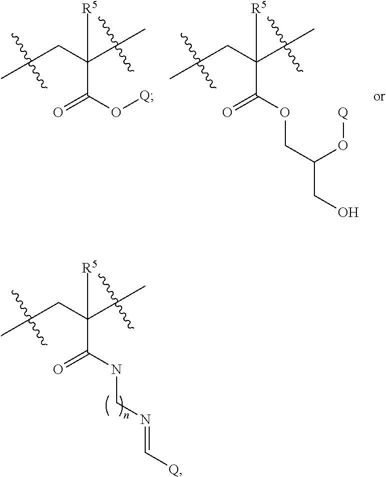

wherein:

R⁵ is, at each occurrence, independently H or C₁-C₆ alkyl;

Q is the outer surface of the solid support; and

n is an integer from 2 to 10.

The B subunit comprises a hydrophilic moiety. The number and identity of the B subunits is controlled to provide the solid supports with the desired hydrophobicity and water contact angles, etc. In certain embodiments, the present inventors have discovered that polymers without B subunits provide certain advantages. Accordingly, certain embodiments are directed to solid supports having polymers which do not comprise B subunits.

In other embodiments, the polymers comprise at least one B subunit. For example, in some embodiments the hydrophilic moiety comprises, at each occurrence, independently amido, ester or hydroxyl functional groups, or combinations thereof.

In other embodiments, the B subunit has, at each occurrence, independently one of the following formulas:

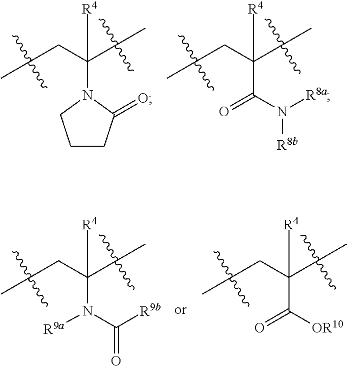

wherein:

R⁴ is at each occurrence, independently H or C₁-C₆ alkyl;

R^{8a} and R^{8b} are each independently H, C₁-C₆ alkyl or hydroxylalkyl;

R^{9a} and R^{9b} are each independently H, C₁-C₆ alkyl or hydroxylalkyl or R^{9a} and R^{9b}, together with the nitrogen atom to which they are bound, join to form a heterocyclic ring; and

R¹⁰ is hydroxylalkyl.

In certain variations of the above, R^{8a} and R^{8b} are each H. In some embodiments, one of R^{8a} or R^{8b} is H, and the other of R^{8a} or R^{8b} is C₁-C₆ alkyl. In still other embodiments, R^{8a} is H, and R^{8b} is methyl.

In certain other embodiments, each hydrophilic moiety has one of the following structures:

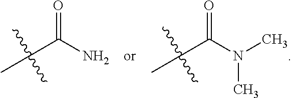

For example, in some embodiments, each hydrophilic moiety has the following structure:

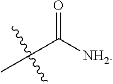 [SMILES: *C(N)=O]

In other embodiments, each hydrophilic moiety has the following structure:

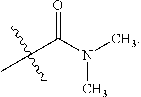 [SMILES: *C(=O)N(C)C]

In still more embodiments, one of R^{8a} or R^{8b} is H, and the other of R^{8a} or R^{8a} is hydroxylalkyl. In other embodiments, R^{8a} is H, and R^{8b} is —CH₂OH.

In other embodiments, at least one hydrophilic moiety has one of the following structures:

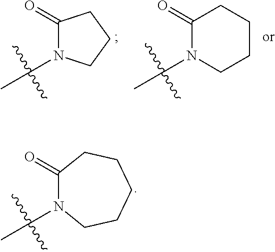 [SMILES: *N1CCCC1=O ; *N1CCCCC1=O ; *N1CCCCCC1=O]

In still other embodiments, R¹⁰ is —CH₂CH₂OH.

The linking moiety which links the first or second thermochemically reactive groups, the hydrophilic moiety or “W” to the rest of the polymer is not limited and can be modified to provide a solid substrate having the desired properties. In certain embodiments, L¹, L² and L³ each independently comprise alkylene, ester, alkylene oxide, amide, imide, ether or dithio moieties, or combinations thereof.

In some embodiments, at least one of L¹, L² or L³ is a direct bond. In other embodiments, each of L¹, L² and L³ are a direct bond.

In various embodiments of the foregoing, at least one of R³, R⁴ or R⁵ is H. For example, in some embodiments each of R³, R⁴ and R⁵ is H. In other embodiments, at least one of R³, R⁴ or R⁵ is methyl. For example, in some embodiments each of R³, R⁴ and R⁵ is methyl.

As noted above, the amount of B subunit in the polymer (and conversely the amount of A subunit) is generally controlled to provide the desired hydrophilicity (and water contact angle) of the resulting solid support. The amount of subunits in the polymer can be expressed as a percentage of the molar feed ratio (MFR %) or as a molar percent. Generally, the molar feed ratio percent will be based on the actual ratio of monomers used for preparation of the polymers. The mole % of subunits can be determined using other techniques, such NMR (e.g., ¹⁹F NMR described herein).

Accordingly, in some embodiments the polymer comprises less than about 40 mol % of B subunits. In other embodiments, the polymer comprises from greater than 0 mol % to about 40 mol % of B subunits. In still other embodiments, the polymer comprises about 35 mol % of B subunits. In some more embodiments, the polymer comprises at least about 30 mol % of B subunits. In other embodiments, the polymer comprises from greater than 0 mol % to about 15 mol % of B subunits.

In still other embodiments, the polymer comprises at least about 75 mol % of A subunits. For example, in some embodiments the polymer comprises at least about 90 mol % of A subunits. In other embodiments, the polymer comprises at least about 95 mol % of A subunits. In still other embodiments, the polymer comprises at least about 99.9 mol % of A subunits.

Accordingly, in some embodiments the polymer comprises less than about 40 MFR % of B subunits. In other embodiments, the polymer comprises from greater than 0 MFR % to about 40 MFR % of B subunits. In still other embodiments, the polymer comprises about 35 MFR % of B subunits. In some more embodiments, the polymer comprises at least about 30 MFR % of B subunits. In other embodiments, the polymer comprises from greater than 0 MFR % to about 15 MFR % of B subunits.

In still other embodiments, the polymer comprises at least about 75 MFR % of A subunits. For example, in some embodiments the polymer comprises at least about 90 MFR % of A subunits. In other embodiments, the polymer comprises at least about 95 MFR % of A subunits. In still other embodiments, the polymer comprises at least about 99.9 MFR % of A subunits.

Embodiments wherein the polymer comprises only one type of reactive group are envisioned within the scope of the invention. Accordingly, in one embodiment, each A subunit comprises the first thermochemically reactive group or the covalent bond to the capture probe. In specific embodiments of the foregoing, the polymer does not comprise B subunits. In even more specific embodiments of the foregoing the first thermochemically reactive group is a reactive ester as defined above for example pentafluorophenyl ester.

In certain embodiments, the polymer is a random polymer.

In certain other embodiments, after spotting (and covalent biomolecule/capture probe attachment) it is desirable to chemically modify the entire remaining reactive polymer surface (non-spotted and non-capture probe areas) so as to render it hydrophilic as well as chemically stable.

Applicants have discovered that the present solid supports have an unexpected ability to switch water contact angles relative to currently available solid supports. That is, the solid supports have a high WCA prior to bioconjugation, which allows for closer spot spacing (e.g., by allowing for decreased spot size). After bioconjugation, the WCA can be significantly decreased by “capping” as explained herein. This decreased WCA after bioconjugation has certain advantages not realized by available solid supports. For example, a more hydrophilic surface facilitates dispensation and dispersion of an aqueous solutions of PCR reagents prior to lyophilization, and other related advantages. The WCA switching ability of the solid supports is discussed in more detail below.

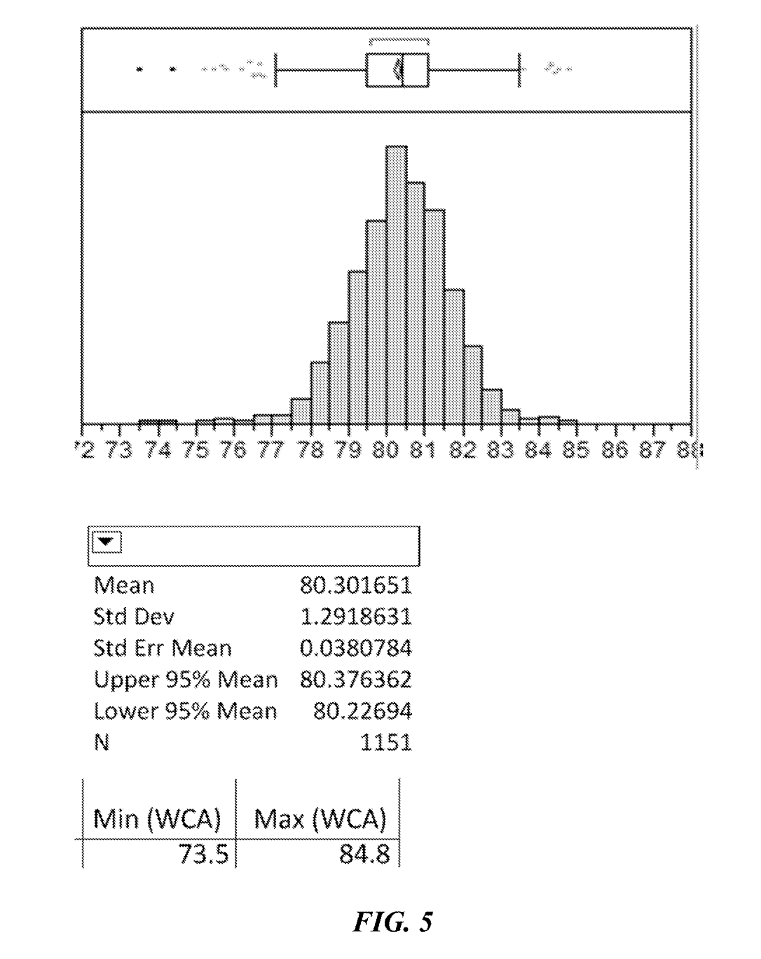

Applicants have unexpectedly discovered that the polymer surface atop the disclosed substrate is more hydrophobic than commercial microarrays currently marketed. FIG. 5 illustrates the WCA of cyclic olefin substrate surfaces immobilized covalently with poly(PFPA-co-DMA) comprising 68.3 mol % of PFPA and 31.7 mol % of DMA. No WCA of less than 73 degrees was observed. The relatively high hydrophobicity prevents the spotted aqueous droplet of capture probe solution on its surface from increasing in diameter due to wetting, enabling the fabrication of closely spaced microarrays.

After spotting the microarray, the remaining reactive groups, which can be hydrophobic (e.g., PFPA) on the surface need to be converted to a hydrophilic moiety by “capping”, which results in WCA≦12° for the overall surface for certain embodiments. The advantages of having such a hydrophilic surface include (1) reducing non-specific adsorption, resulting in high signal to noise ratio, (2) enabling the dispensed aqueous solution of lyophilized reagents to spread uniformly on the surface prior to lyophilization, (3) expelling entrapped air bubbles during the reconstitution of lyophilized reagents with aqueous buffer.

A variety of surface treatments to effect capping were tested, including aqueous triethylamine (TEA), aqueous ammonia, ammonia vapor, capping by immersion with short PEG diamines, capping with a long PEG amine (MW 2000), capping with a short PEG diamine vs. ammonia for one hour immersion time in acetone vs. water, salt vs. no salt and at 40° C. and 60° C., capping with a short PEG diamine vs. ammonia with 100 mM triethylamine (TEA) at 20° C., 60° C. and 95° C. for one hour, capping with dimethylamine at three concentrations (50, 150, and 500 mM), in water containing 50 mM TEA vs. no TEA, for one hour at 60° C. vs. 75° C., and capping with ammonia at four concentrations (0, 50, 100, 500 mM), for one hour at each of four temperatures (20° C., 60° C., 75° C. and 95° C.).

Table 1 (see example 12) presents exemplary capping results for solid supports prepared by covalent immobilization of a copolymer having 65 mol % PFPA and 35 mol % DMA onto substrate surfaces previously treated with atmospheric pressure oxygen plasma. Ammonia capping converts PFPA monomer repeating units having hydrophobic perfluorinated ester groups to hydrophilic and chemically stable acrylamide groups. As illustrated in Table 1, following capping by immersion of the spotted microarray in 50-500 mM aqueous ammonia, 100 mM triethylamine for 1-2 hr. at 60° C. produced water contact angles below 10 degrees.

Applicants have unexpectedly discovered that ammonia is uniquely well suited to switching the WCA water contact angle from about 85 degrees to <20 degrees, or even less than 15 degrees or less than 10 degrees. Applicants have unexpectedly found that the above capping protocol was one way to convert the water contact angle of the spotted (i.e., capture probe bound) solid support from about 80° degrees to ≦15° degrees. The low water contact angle of ≦15° reduced non-specific adsorption and increased the signal to noise ratio thus increasing sensitivity and specificity when detecting the probe signal. The high aqueous wettability of the capped surface provides a hydrophilic surface useful for integration into a microfluidic device and assists in reducing the nonspecific adsorption of various bioassay components and air bubbles.

In other embodiments, the water contact angle is optimized to obtain small spot sizes (e.g., when the solid support is used in array-type analyses for high degree of multiplexing). In some embodiments, the solid support has a water contact angle ranging from 40° to 95°, for example from 40° to 90°, from 60° to 95° or from 70° to 90°. For example, in some embodiments the solid support has a water contact angle ranging from 50° to 85° or from 60° to 85°. In other embodiments, the solid support has a water contact angle ranging from 60° to 80°. In other embodiments, the solid support has a water contact angle ranging from 61° to 95°, for example from 70° to 90°. For example, in some embodiments the solid support has a water contact angle ranging from 75° to 85°. In other embodiments, the solid support has a water contact angle ranging from 78° to 83°.

In some embodiments, the WCA after an optional capping step (e.g., treatment with ammonia) is much lower than before capping. In some embodiments, the WCA after capping is less than 25°, less than 20°, less than 15° or even less than 10°. The difference in WCA before and after an optional capping step is, in some embodiments, at least 50°, at least 60° or at least 70°.

The solid substrate employed in the solid supports herein is not limited and is generally chosen based upon the desired end use. However, the present inventors have discovered that certain embodiments of the solid supports can be employed with organic polymer substrates. In some embodiments, the substrate comprises poly(styrene), poly(carbonate), poly(ethersulfone), poly(ketone), poly(aliphatic ether), poly(ether ketone), poly(ether ether ketone), poly(aryl ether), poly(amide) poly(imide), poly(ester) poly(acrylate), poly(methacrylate), poly(olefin), poly(cyclic olefin), poly(vinyl alcohol), polymer blends or poly alkyl polymers or halogenated derivatives, crosslinked derivatives or combinations thereof. For example, in some embodiments the halogenated derivatives are halogenated poly(aryl ether), halogenated poly(olefin) or halogenated poly(cyclic olefin). In certain specific examples, the substrate comprises a cyclic poly(olefin).

In some more embodiments, the substrate is substantially optically transparent. Such substrates find utility in solid supports employed in analyses using fluorescent or optical detection methods. In some embodiments, the substrate is substantially optically transparent between about 400 nm and about 800 nm. In still other embodiments, the substrate is at least about 90% optically transparent.

As noted above, the solid supports may be used in methods for array analysis of various analytes, such as DNA. Accordingly, in some embodiments the solid support comprises a systematic array of distinct locations, each distinct location independently comprising at least one of the polymers covalently bound to the outer surface of the substrate. In other embodiments, each distinct location independently comprises a plurality of the polymers covalently bound thereto. In still other embodiments, at least one polymer at each distinct location independently comprises a capture probe covalently bound thereto. For example, in some embodiments each distinct location comprises a plurality of structurally distinct capture probes bound thereto.

In contrast to previously described solid supports, the embodiments of the presently described solid supports comprise substantially no chemical cross links (inter and intra polymer cross-links) between the plurality of polymers. While not wishing to be bound by theory, the present inventors believe such inter and intra polymer cross-links are formed during UV induced bonding of photoactive polymers to substrates (via UV-induced radical mechanisms). Since embodiments of the present polymers are covalently bound to the solid substrates via thermochemically reactive functional groups (i.e., not UV reactive functional groups) the resulting solid supports generally comprise substantially no inter or intra polymer cross-links.

Accordingly, in some embodiments the plurality of polymers is substantially free of cross links therebetween. In other embodiments, the plurality of polymers is 95%, 98%, 99% or even 99.9% free of cross links therebetween

The present disclosure also provides certain solid substrates which have been found useful in the preparation of the solid supports described above. For example, in one embodiment the disclosure provides a solid support comprising a plurality of primary amine functional groups covalently bound to an outer surface of the solid substrate, wherein the amine functional groups are bound to the solid substrate through a linker comprising an imine bond.

In certain embodiments of the above solid support, an outer surface of the solid substrate has the following structure:

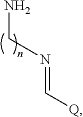

or a salt, tautomer or stereoisomer thereof, wherein:

Q is the outer surface of the solid substrate; and

n is an integer from 2 to 10.

It is understood that any embodiments of the compounds and/or polymers, as set forth herein, and any specific substituent set forth herein in the compounds and/or polymers described herein, may be independently combined with other embodiments and/or substituents of the compounds and/or polymers described herein to form embodiments of the inventions not specifically set forth above. In addition, in the event that a list of substituents is listed for any particular R group in a particular embodiment and/or claim, it is understood that each individual substituent may be deleted from the particular embodiment and/or claim and that the remaining list of substituents will be considered to be within the scope of the invention.

It is understood that in the present description, combinations of substituents and/or variables of the depicted formulae are permissible only if such contributions result in stable compounds.

Furthermore, all compounds and/or polymers of the invention which exist in free base or acid form can be converted to salts by treatment with the appropriate inorganic or organic base or acid by methods known to one skilled in the art. Salts of the compounds of the invention can be converted to their free base or acid form by standard techniques.

### B. Methods for Preparation of the Solid Supports and Polymers

Embodiments of the present invention are directed to methods for preparation of the solid supports. For example, in one embodiment the method comprises:

A) providing a solid substrate comprising a plurality of hydroxyl, carbonyl or amine functional groups, or combinations thereof covalently bound to the outer surface thereof; and

B) contacting a polymer comprising D and optional E and F subunits with the solid substrate under conditions sufficient to form a covalent bond between at least one of the hydroxyl, carbonyl or amine functional groups and the D subunit, wherein:

the D subunit, at each occurrence, independently comprises a first reactive group, wherein the first reactive group is a thermochemically reactive group capable of forming a covalent bond with an alcohol, carbonyl or amine functional group on a solid substrate or capture probe;

the E subunit, at each occurrence, independently comprises a hydrophilic moiety; and

the F subunit, at each occurrence, independently comprises a second reactive group, wherein the second reactive group is a cycloaddition or conjugate addition reactive group having a reactivity specific for covalent bond formation with a target functional group on a capture probe via a cycloaddition or 1,4-conjugate addition reaction,

wherein the reactivity of the first reactive group and the second reactive group are orthogonal to each other.

In certain embodiments of the foregoing, the hydroxyl and carbonyl functional groups are bound directly to the substrate surface without intervening linkers, and the amine functional groups are bound to the substrate surface through a linker comprising an imine bond, the imine bond being bound directly to the substrate surface without an intervening linker. In some embodiments, the amine functional groups are bound to the solid substrate without an intervening linker.

In certain embodiments, the methods for preparation of the solid supports comprise reacting a reactive polymer with a substrate surface which has been activated as described above to contain hydroxyl, epoxide, aldehyde, acid, amine or other functional groups. In some embodiments, the reactive polymer comprises A subunits as described above and optional B subunits. Upon reaction with the functional groups on the substrate surface, the A subunits are converted to C subunits. The remaining, unreacted A subunits are available for bioconjugation with a capture probe.

In other embodiments of the foregoing method, the method further comprises a capping step. The capping step may be performed after conjugation of a capture probe to the solid support and generally results in a solid support having a significantly lower WCA as discussed above. Useful reagents for the optional capping step include bases, such as amine bases (e.g., NH₄OH). Amine-containing catalysts may also be employed to facilitate the reaction. Useful solvents include polar solvents, such as acetonitrile and/or acetone, which may be anhydrous or include a small proportion of water. Capping may be performed at room temperature, but will typically be performed at elevated temperatures such as about 60° C., 75° C. or 95° C.

Optionally, the present methods may include use of a catalyst (e.g., basic catalyst) to improve the reaction of the polymer with the solid substrate.

In some embodiments, the first reactive group is a nucleophilic group capable of covalent bond formation with a ketone or aldehyde group on the solid substrate. For example, in some embodiments the first reactive group is a hydrazide, amine or alkoxyamine.

In other embodiments, the first reactive group is an electrophilic group capable of covalent bond formation with an alcohol or amine group on the solid substrate. For example, in some embodiments the first reactive group is an aryl ester or an epoxide.

In some other embodiments, the polymer has the following structure (III):

T³-(D)ₐ(E)_{b}(F)_{c}T⁴ (III)

wherein:

D, E and F represent the D, E and F subunits, respectively;

T³ and T⁴ are each independently absent or polymer terminal groups selected from H, alkyl and an initiator residue;

a is an integer from 1 to 50,000; and

b and c are independently an integer from 0 to 50,000.

In other embodiments of the foregoing method, the polymer has the following formula (IV):

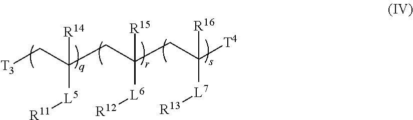

wherein:

R¹¹ is, at each occurrence, independently a substituent comprising the first reactive group;

R¹² is, at each occurrence, independently a substituent comprising the hydrophilic moiety;

R¹³ is, at each occurrence, independently a substituent comprising the second reactive group;

R¹⁴, R¹⁵ and R¹⁶ are, at each occurrence, independently H or C₁-C₆ alkyl;

L⁵, L⁶ and L⁷ are, at each occurrence, independently a direct bond or a linker up to 100 atoms in length;

T³ and T⁴ are each independently absent or polymer terminal groups selected from H, alkyl and an initiator residue;

q is an integer from 1 to 50,000; and

r and s are independently an integer from 0 to 50,000.

In some other exemplary embodiments, R¹¹ has, at each occurrence, independently one of the following formulas:

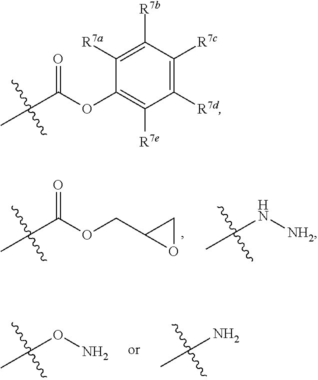

wherein R^{7a}, R^{7b}, R^{7c}, R^{7d} and R^{7e} are each independently H, halo, trihalomethyl, or nitro.

In certain of the above embodiment, r and s are each 0.

In some embodiments, the thermochemically reactive group is as defined in any of the embodiments herein above.

In some more embodiments, the F subunit is present. In other embodiments, the cycloaddition or conjugate addition reactive group is as defined in any of the embodiments herein above.

In other embodiments, the E subunit is present. In some of these embodiments, the hydrophilic moiety is as defined in any of the embodiments herein above.

In certain other examples, the covalent bond is an ether, ester, hydrazone, oxime, amide or imine bond formed by reaction of at least one of the hydroxyl, amine or carbonyl moieties with the first reactive group. In other examples, W comprises an ether, ester, hydrazone, oxime or imine bond formed by reaction of at least one of the hydroxyl or carbonyl moieties with the first reactive group

In some more embodiments, the solid substrate is prepared by corona treatment or treating the solid substrate with ambient air plasma, atmospheric pressure oxygen plasma, (APOP), nitrogen plasma, ammonia plasma or a mixture of nitrogen+hydrogen plasma. For example, in some embodiments the method further comprises contacting the solid substrate with a diamine compound under conditions sufficient to form a covalent imine bond between a carbonyl on the solid substrate and a first amine group in the diamine.

In other embodiments, the method further comprises contacting the solid support with a capture probe under conditions sufficient to form a covalent bond between the capture probe and the polymer.

In still more embodiments, the covalent bond is formed by reaction of an aryl ester or epoxide moiety on the D subunit and an amine moiety on the capture probe.

In other embodiments, the covalent bond is formed by reaction of an alkyne moiety on the F subunit and an azide moiety on the capture probe. In other embodiments, the covalent bond is formed by reaction of an azide moiety on the F subunit and an alkyne moiety on the capture probe. For example, certain embodiments of the method further comprise contacting a Cu(I) catalyst with the solid support in the presence of an azide.

Methods for preparation of the disclosed solid supports and polymers will be readily apparent to one of ordinary skill in the art. For example, in certain embodiments polymers of the present invention may be prepared by admixing the desired ratio of subunits and an optional activator (e.g., AIBN for thermal polymerization or a catalyst for ATRP). Subunits and polymers comprising click functional groups, such as azide or alkynes can be prepared according to methods known in the art or purchased from commercial sources (e.g., propargyl acrylate or 3-azidopropylacrylate). See e.g., S. R. Gondi, el at., Macromolecules 2007, 40, 474-481; P. J. Roth, el at., J. Polym. Sci. Part A: Polym. Chem. 2009, 47, 3118-3130; and C. Li, et al., Macromolecules, 2009, 42, 2916-2924, the disclosures of which are hereby incorporated by reference in their entirety. Exemplary methods are provided in the examples.

It will also be appreciated by those skilled in the art that in the processes described herein the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (for example, t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R″ (where R″ is alkyl, aryl or arylalkyl), p-methoxybenzyl, trityl and the like. Suitable protecting groups for carboxylic acid include alkyl, aryl or arylalkyl esters. Protecting groups may be added or removed in accordance with standard techniques, which are known to one skilled in the art and as described herein. The use of protecting groups is described in detail in Green, T. W. and P. G. M. Wutz, Protective Groups in Organic Synthesis (1999), 3rd Ed., Wiley. As one of skill in the art would appreciate, the protecting group may also be a polymer resin such as a Wang resin, Rink resin or a 2-chlorotrityl-chloride resin.

### C. Polymers

In another aspect, the present invention is directed to novel polymers. The polymers can be used for preparation of the described solid support or for other purposes. Polymers containing acrylamide are generally thought to be soluble only in aqueous phases. However, in contrast to the general teachings of the art, the present inventors have unexpectedly discovered that a copolymer of acrylamide and a hydrophobic acrylate monomer is appreciably soluble in organic solvent. In this regard, the present inventors have discovered that incorporation of a small fraction of acrylamide in a copolymer with a hydrophobic monomer, e.g. PFPA, yields a copolymer which has advantageous properties. One advantage of such a copolymer is that solubility in various solvents (including water) may be tuned for functional surface applications by adjusting the acrylamide monomer content. This also may provide a copolymer with the highest percentage of reactive functional groups while preserving enough hydrophilic functionality for utility in subsequent water-based assays. Surprisingly, in certain embodiments, exemplary polymers having an acrylamide MFR of less than 35% have been found to be readily soluble in acetone, acetonitrile, THF, chloroform and other organic solvents.

In one embodiment, the polymer comprises G, H and optional I subunits, wherein:

the G subunit, at each occurrence, independently comprises:
a) a first thermochemically reactive group, wherein the first thermochemically reactive group is capable of forming a covalent bond with an alcohol, carbonyl or amine group;
b) a second thermochemically reactive group, wherein the second thermochemically reactive group is a cycloaddition or conjugate addition reactive group having a reactivity specific for covalent bond formation with a target functional group via a cycloaddition or 1,4-conjugate addition reaction;

the H subunit, at each occurrence, has the following structure:

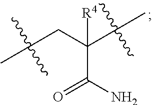

and

the optional I subunit, at each occurrence, independently comprises a hydrophilic moiety and has one of the following structures:

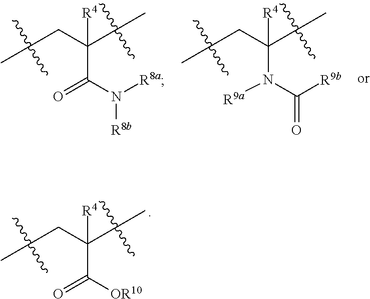

wherein:

R⁴ is at each occurrence, independently H or C₁-C₆ alkyl;

R^{8a} is H, C₁-C₆ alkyl or hydroxylalkyl;

R^{8b} is C₁-C₆ alkyl or hydroxylalkyl

R^{9a} and R^{9b} are each independently H, C₁-C₆ alkyl or hydroxylalkyl or R^{9a} and R^{9b}, together with the nitrogen atom to which they are bound, join to form a heterocyclic ring; and

R¹⁰ is hydroxylalkyl,
wherein the reactivity of the first and second thermochemically reactive groups are orthogonal to each other.

In certain embodiments, the optional I subunit is absent. In other embodiments, the optional I subunit is present.

In some embodiments, the hydrophilic moiety is as defined in any of the embodiments herein above.

In other embodiments, the G subunit comprises the first and/or second thermochemically reactive group as defined in any of the embodiments herein above with respect to the A subunit. In certain embodiments, each G subunit comprises the first thermochemically reactive group.

In various embodiments, the polymer comprises from greater than 0 mol % to about 15 mol % of H subunits. In other various embodiments, the polymer comprises from greater than 0 MFR % to about 15 MFR % of H subunits.

### D. Methods of Use of the Solid Substrates

Certain embodiments of the present invention are directed to methods. Such methods include, but are not limited to methods for preparation of the polymers, activated solid substrates and solid supports described herein. Methods for use of the solid supports in analytical assays are also provided. For example, the solid supports may be used in assays for the detection of any number of analytes, for example viruses, bacteria, plasmodium, fungi, as well as metals and unknown bio-warfare, bio-hazard and chemical warfare materials.

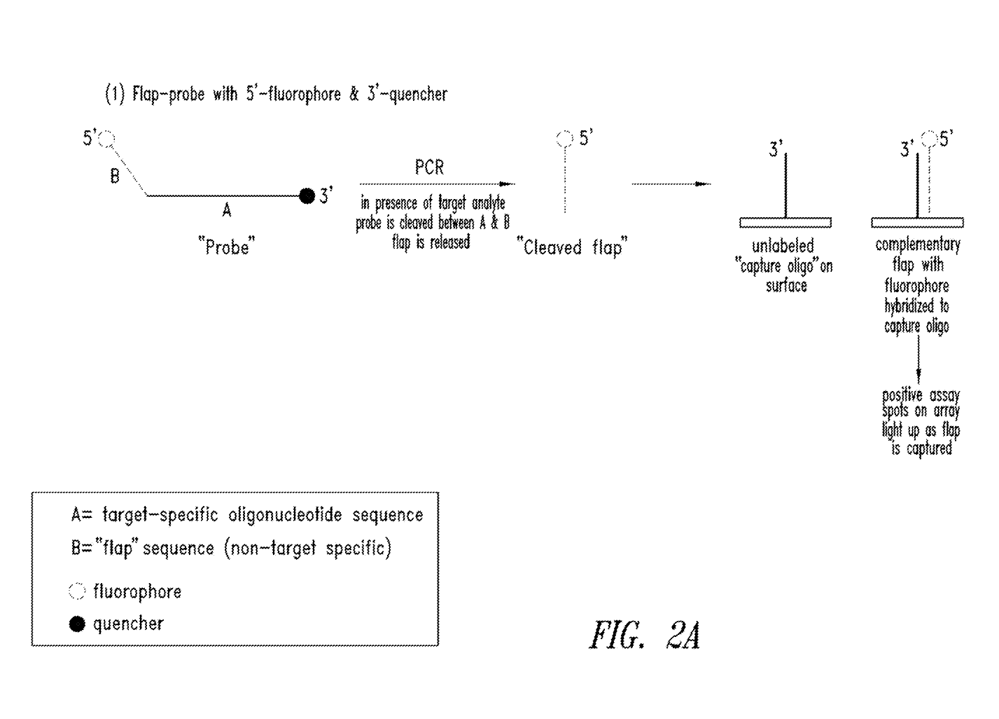

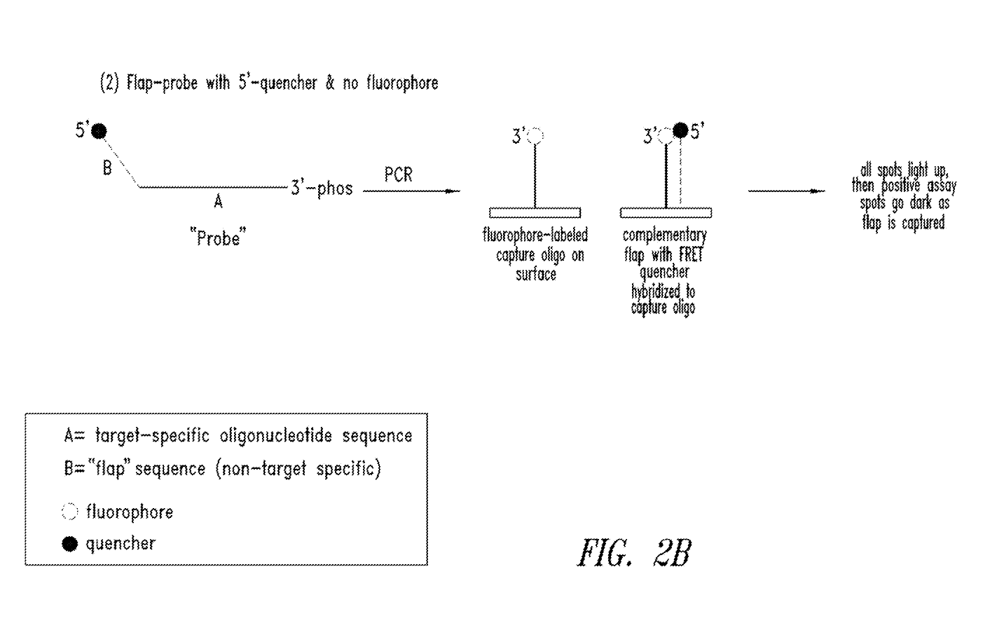

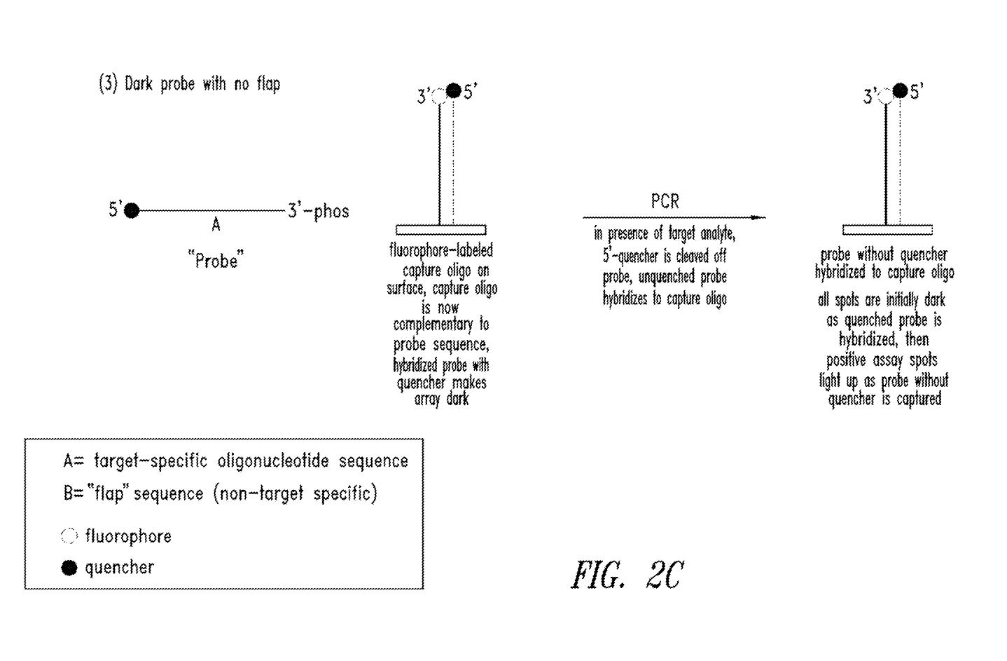

Methods for use of the solid supports for analysis of various analytes will be apparent to one of ordinary skill in the art. Such methods are described for example in Provisional U.S. Patent Application Nos. 61/463,580, 61/561,198, 1/684,104, 61/600,569, U.S. patent application Ser. No. 13/399,872 and U.S. Pub. No. 2012/0214686, the full disclosures of which are hereby incorporated herein by reference in their entirety for all purposes. Exemplary methods for use of the disclosed solid supports are depicted in schematically in FIG. 2.

As depicted in FIG. 2A, in one embodiment of the methods an analyte probe comprises sections A and B. The A section optionally comprises a quencher moiety, the quencher may be at the 3′ end of the A section or at any other point within the A section. The A section is complementary to at least a portion of a target analyte sequence (e.g., pathogen DNA, etc.). The analyte probe also comprises section B (the “flap”). The flap comprises a fluorophore and a sequence complementary to at least a portion of a sequence of a capture probe bound to the solid support. Optionally, the sequence of the analyte probe is selected such that the A section and the flap have at least some complementarity so that the quencher and fluorophore are brought into close proximity, thus decreasing the fluorescent signal associated with the unbound analyte probe and increasing the overall sensitivity of the assay.

The assay conditions generally include a plurality of analyte probes having unique sequences specific for different target analytes. Under PCR conditions, and in the presence of a complementary (or at least partially complementary) target analyte, the flap is cleaved from the analyte probe. The cleaved flap is then hybridized to a solid support-bound capture probe complementary (or at least partially complementary) to the flap. The presence (or increase) of a fluorescent signal at the position to which the capture probe is bound indicates the presence of the target analyte sequence.

An alternate embodiment is depicted in FIG. 2B. In this exemplary embodiment, the flap comprises a quencher and the support bound capture probe comprises a fluorophore. Again, the exact position of the quencher or fluorophore on the flap or capture probe, respectively, can be varied. Under PCR conditions in the presence of the target analyte sequence, the flap is cleaved from the probe. The flap is then hybridized to the capture probe and the fluorophore on the capture probe is thereby quenched. Accordingly, the absence (or decrease) of a fluorescent at the position which the capture probe is bound indicates the presence of the target analyte sequence.

Yet another exemplary method is provided in FIG. 2C. Here, the probe comprises a sequence which is at least partially complementary to a target analyte sequence and does not comprise a cleavable flap. The probe in this embodiment comprises a quencher and the support-bound capture probe comprises a fluorophore. The probe is hybridized with the capture probe, resulting in a quenched signal at the position to which the capture probe is bound. The solid support is then subjected to PCR conditions. In the presence of the target analyte sequence, the probe quencher is cleaved off and the fluorescent signal from the capture probe increases.

Accordingly, in one embodiment, the invention is generally directed to a method for determining the presence or absence of a target analyte molecule, the method comprising:

a) providing a solid support as described herein, wherein the A subunit comprises a capture probe covalently bound thereto;

b) contacting an analyte probe or fragment thereof with the solid support; and

c) detecting the presence or absence of a signal produced from interaction of the capture probe with the analyte probe.

In certain embodiments of the foregoing, the capture probe is a polynucleotide. In some more embodiments, the target analyte molecule is a polynucleotide or a protein.

In other embodiments, the signal is a fluorescent signal. For example, in some embodiments the fluorescent signal is produced or reduced as a result of specific hybridization of the analyte probe with a capture probe.

In other embodiments, the analyte probe comprises a fluorophore or a fluorophore quencher.

In other related embodiments, the invention provides a method of detecting a target nucleic acid, the method comprising:

A) providing a detection chamber comprising at least one solid support described herein, the solid support comprising an array of capture probes;

B) loading a sample into the detection chamber, which sample comprises one or more copies of the target nucleic acid to be detected;

C) hybridizing an amplification primer and a probe to the one or more copies;

D) amplifying at least a portion of one or more of the target nucleic acid copies in an amplification primer dependent amplification reaction, wherein the amplification reaction results in cleavage of the probe and release of a first probe fragment;

E) hybridizing the first probe fragment to the high-efficiency array; and,

F) detecting a signal produced by binding the first probe fragment to the array, thereby detecting the target nucleic acid.

In certain embodiments, the detecting step(s) is carried out under conditions that reduce background signal proximal to the array.

In other embodiments, the methods comprise analyzing a sample for a plurality of target nucleic acid sequences, the method comprising:

A) contacting the sample with a first plurality of labeled probes, each of the first plurality of labeled probes comprising a first portion complementary to a different target sequence of interest in a first panel of target nucleic acid sequences and a second portion complementary to a different capture probe on a high efficiency probe array, the high efficiency probe array comprising a solid support as described herein, wherein the second portion has a label attached thereto and is not complementary to the target sequence of interest;

B) amplifying any target sequences from the first panel of target nucleic acid sequences that are present in the sample, in an amplification primer dependent amplification reaction, wherein the amplification reaction results in cleavage of labeled probes hybridized to the target sequences and release of the second portion of the labeled probes bearing the label;

C) hybridizing the released second portion of the labeled probes to the high-efficiency array;

D) detecting binding of the second portion of the labeled probe to a capture probe in the high efficiency array; and

E) identifying the target sequences present in the sample from the second portions of the labeled probes that hybridize to the high efficiency array.

In still other embodiments, the invention provides a method of detecting the presence of a target nucleic acid sequence in a sample, the method comprising:

A) performing an amplification reaction on the sample with a polymerase enzyme that possesses nuclease activity, in the presence of a first labeled probe that comprises a first portion complementary to a first target nucleic acid sequence and a second labeled portion not complementary to the first target nucleic acid sequence, such that the second portion is cleaved from the first portion when the target nucleic acid sequence is amplified;

B) hybridizing the second labeled portion to a capture probe complementary to the second portion; the capture probe being covalently bound to a solid support described herein; and

C) detecting the presence of the second labeled portion hybridized to the capture probe on the substrate.

Still other embodiments of the methods comprise a method of detecting a target nucleic acid sequence in a sample, the method comprising:

A) performing an amplification reaction on the sample with a polymerase enzyme that possesses nuclease activity, in the presence of a reagent comprising first probes that comprise a first portion complementary to the target nucleic acid sequence and a second portion not complementary to the first target nucleic acid sequence, the second portion comprising a first quencher moiety coupled to the second portion at a first position, such that the second portion is cleaved from the first portion as a first probe fragment, when the target nucleic acid sequence is amplified;

B) hybridizing the first probe fragment to capture probes immobilized upon a solid support described herein, wherein the capture probes comprise a fluorophore that is at least partially quenched by the first quencher moiety, the fluorophore coupled to a second position on the capture probes such that upon hybridization of the probe fragments to the capture probes, the fluorophore is at least partially quenched by the quencher; and

C) detecting the presence of the target sequence based upon the quenching of the fluorophore on the capture probes.

In another embodiments, the invention is directed to a method of detecting the presence of at least a first target nucleic acid sequence in a sample, the method comprising:

A) subjecting the sample to an amplification reaction capable of amplifying the target nucleic acid sequence in the presence of a solid support described herein, wherein the solid support comprises at least a first set of nucleic acid probes, the first set of nucleic acid probes comprising a capture probe comprising a fluorophore attached thereto, and a target specific nucleic acid probe complementary to at least a portion of the capture probe and the target nucleic acid sequence and comprising a quencher attached thereto, such that the quencher quenches fluorescence from the fluorophore when the target specific probe is hybridized to the capture probe; and

B) detecting fluorescence from the sample following one or more cycles of the polymerase chain reaction, an increase in fluorescence being indicative of the presence of the target nucleic acid sequence.

The present invention also provides devices and consumables comprising the solid supports and solid substrates described herein. In one embodiments, the invention provides a nucleic acid detection device, the nucleic acid detection device comprising:

A) a detection chamber that comprises at least one high efficiency nucleic acid detection array on at least one surface of the chamber, the nucleic acid detection array comprising a solid support described herein, wherein the chamber is configured to reduce signal background for signals detected from the array;

B) a thermo-regulatory module operably coupled to the detection chamber, which module regulates temperature within the chamber during operation of the device; and,

C) an optical train that detects a signal produced at the array during operation of the device.

In other embodiments, the invention provides a nucleic acid detection consumable, the nucleic acid detection consumable comprising: a thin chamber less than about 500 μm in depth, which chamber comprises an optically transparent window that comprises a high efficiency capture nucleic acid array disposed on an inner surface of the window, which chamber additionally comprises at least one reagent delivery port fluidly coupled to the chamber, wherein the consumable is configured to permit thermocycling of fluid within the chamber, wherein the high efficiency capture nucleic acid array comprises a solid support described herein.

In certain embodiments, the target analyte molecule is a DNA sequence, the DNA sequence having a sequence which indicates the presence of a pathogen, for example a virus, bacteria, plasmodium or fungus.

In some embodiments, the analyte probe is a flap. In some other embodiments, the analyte probe comprises a quencher. In some other embodiments, the analyte probe comprises a fluorophore. In still other embodiments, the capture probe comprises a fluorophore. In still other embodiments, the probe comprises an oligonucleotide.

The solid support may be any of the solid supports described herein. Further, in certain embodiments, the capture probe is a polynucleotide, and in other embodiments the target analyte molecule is a polynucleotide. In still other embodiments, the target analyte molecule is prepared via a polymerase chain reaction.

In some other embodiments, the signal is a fluorescent signal. For example, in some embodiments the fluorescent signal is produced as a result of specific hybridization of a target analyte molecule with a capture probe.

In other related embodiments, the invention provides a method for detecting an analyte in a sample. The method includes contacting the analyte with a solid support of the invention to allow capture of the analyte by the capture probe of the solid support of the invention and detecting capture of the analyte. In certain embodiments, the analyte is a biomolecule, such as a polypeptide, a nucleic acid, a carbohydrate, a lipid, or hybrids thereof. In other embodiments, the analyte is an organic molecule such as a drug, drug candidate, cofactor or metabolite. In another embodiment, the analyte is an inorganic molecule, such as a metal complex or cofactor. In an exemplary embodiment, the analyte is a nucleic acid which is a labeled probe. In another exemplary embodiment, the invention provides a reactive surface that covalently immobilizes a protein, an enzyme, an antibody, an antigen, a hormone, a carbohydrate, a glycoconjugate or a synthetically produced analyte target such as synthetically produced epitope that may be used to capture and detect an analyte in a subsequent step.

In various other embodiments, the invention provides a method of detecting a target nucleic acid using a solid support of the invention. The methods include binding a detectably labeled nucleic acid probe fragment to a nucleic acid of complementary sequence immobilized on the polymer of the solid support of the invention. An exemplary method includes:

A) hybridizing an amplification primer and a detectably labeled probe to the target nucleic acid;

B) amplifying at least a portion of the target nucleic acid in a primer dependent amplification reaction, wherein the amplification reaction results in cleavage of the labeled probe and release of a labeled probe fragment; and

C) hybridizing the labeled probe fragment to the immobilized assay component, wherein said component is a nucleic acid at least partially complementary to said labeled probe fragment, thereby detecting said nucleic acid.

Detection of the analyte can be accomplished by any art-recognized method or device. In certain embodiments, the analyte is detected by a fluorescent signal arising from an analyte or probe immobilized on the solid support. In an exemplary embodiment, the solid support of the invention is a nucleic acid array, and the signal arises from a fluorescently labeled nucleic acid hybridized to an assay component immobilized on the polymer of the solid support. In various embodiments, the immobilized assay component is a nucleic acid with a sequence at least partially complementary to the sequence of the fluorescently labeled nucleic acid. In selected embodiments in which the analyte is fluorescently labeled, it is detected by a fluorescence detector such as a CCD array. In certain embodiments the method involves profiling a certain class of analytes (e.g., biomolecules, e.g., nucleic acids) in a sample by applying the sample to one or more addressable locations of the solid support and detecting analytes captured at the addressable location or locations. Examples of methods useful for implementing the present invention include those described in Provisional U.S. Patent Application No. 61/561,198, and U.S. Ser. No. 13/399,872, the full disclosures of which are hereby incorporated herein by reference in their entirety for all purposes.

In some embodiments, the solid supports of the present invention are useful for the isolation and detection of analytes in an assay mixture. In particular, solid supports of the invention are useful in performing assays of substantially any format including, but not limited to the polymerase chain reaction (PCR), chromatographic capture, immunoassays, competitive assays, DNA or RNA binding assays, fluorescence in situ hybridization (FISH), protein and nucleic acid profiling assays, sandwich assays and the like. The following discussion focuses on the use of a solid support of the invention to practice exemplary assays. This focus is for clarity of illustration only and is not intended to define or limit the scope of the invention. Those of skill in the art will appreciate that the method of the invention is broadly applicable to any assay technique for detecting the presence and/or amount of an analyte.

In various embodiments, the invention provides a method of detecting a target nucleic acid using a solid support of the invention. The methods includes binding a detectably labeled nucleic acid probe fragment to a nucleic acid of complementary sequence immobilized on the reactive polymer of the solid support of the invention. An exemplary method includes:

A) hybridizing an amplification primer and a detectably labeled probe to the target nucleic acid;

B) amplifying at least a portion of the target nucleic acid in a primer dependent amplification reaction, wherein the amplification reaction results in cleavage of the labeled probe and release of a labeled probe fragment; and

C) hybridizing the labeled probe fragment to the immobilized assay component, wherein said component is a nucleic acid at least partially complementary to said labeled probe fragment, thereby detecting said nucleic acid.

A sample can be from any source, and can be a biological sample, such as a sample from an organism or a group of organisms from the same or different species. A biological sample can be a sample of bodily fluid, for example, a blood sample, serum sample, lymph sample, a bone marrow sample, ascites fluid, pleural fluid, pelvic wash fluid, ocular fluid, urine, semen, sputum, or saliva. A biological sample can also be an extract from cutaneous, nasal, throat, or genital swabs, or extracts of fecal material. Biological samples can also be samples of organs or tissues, including tumors. Biological samples can also be samples of cell cultures, including both cell lines and primary cultures of both prokaryotic and eukaryotic cells.

A sample can be from the environment, such as from a body of water or from the soil, or from a food, beverage, or water source, an industrial source, workplace area, public area, or living area. A sample can be an extract, for example a liquid extract of a soil or food sample. A sample can be a solution made from washing or soaking, or suspending a swab from, articles such as tools, articles of clothing, artifacts, or other materials. Samples also include samples for identification of biowarfare agents, for example samples of powders or liquids of known or unknown origin.

A sample can be an unprocessed or a processed sample; processing can involve steps that increase the purity, concentration, or accessibility of components of the sample to facilitate the analysis of the sample. As non-limiting examples, processing can include steps that reduce the volume of a sample, remove or separate components of a sample, solubilize a sample or one or more sample components, or disrupt, modify, expose, release, or isolate components of a sample. Non-limiting examples of such procedures are centrifugation, precipitation, filtration, homogenization, cell lysis, binding of antibodies, cell separation, etc. For example, in some preferred embodiments of the present invention, the sample is a blood sample that is at least partially processed, for example, by the removal of red blood cells, by concentration, by selection of one or more cell or virus types (for example, white blood cells or pathogenic cells), or by lysis of cells, etc.

Exemplary samples include a solution of at least partially purified nucleic acid molecules. The nucleic acid molecules can be from a single source or multiple sources, and can comprise DNA, RNA, or both. For example, a solution of nucleic acid molecules can be a sample that was subjected to any of the steps of cell lysis, concentration, extraction, precipitation, nucleic acid selection (such as, for example, poly A RNA selection or selection of DNA sequences comprising Alu elements), or treatment with one or more enzymes. The sample can also be a solution that comprises synthetic nucleic acid molecules.

In an exemplary embodiment, when the solid support of the invention is used to detect and/or characterize a nucleic acid, the solid support of the invention is a nucleic acid array having a plurality of nucleic acids of different sequences covalently bound to the surface-bound polymer at known locations on the solid support. In various embodiments, the solid support is a component of a reaction vessel in which PCR is performed on a target nucleic acid sample contained in an assay mixture. In an exemplary method, one or more nucleic acid primer and a detectably labeled nucleic acid probe are hybridized to the target nucleic acid. During PCR template extension, the probe is cleaved, producing a probe fragment. The probe fragment is released from the target nucleic acid and is captured by an immobilized analyte component, which is a nucleic acid, on the surface bound polymer. The probe sequence is determined by its binding location on the array.

In various embodiments the solid supports of the invention are utilized as a component of a multiplex assay for detecting one or more species in an assay mixture. The solid supports of the invention are particularly useful in performing multiplex-type analyses and assays. In an exemplary multiplex analysis, two or more distinct species (or regions of one or more species) are detected using two or more probes, wherein each of the probes is labeled with a different fluorophore. The solid supports of the invention allow for the design of multiplex assays in which more than one detectably labeled probe structure is used in the assay. A number of different multiplex assays using the solid supports of the invention will be apparent to one of skill in the art. In one exemplary assay, each of at least two distinct fluorophores is used to signal hybridization of a nucleic acid probe fragment to a surface immobilized nucleic acid.

Exemplary labeled probes of use in practicing the methods of the invention are nucleic acid probes. Useful nucleic acid probes include those that can be used as components of detection agents in a variety of DNA amplification/quantification strategies including, for example, 5′-nuclease assay, Strand Displacement Amplification (SDA), Nucleic Acid Sequence-Based Amplification (NASBA), Rolling Circle Amplification (RCA), as well as for direct detection of targets in solution phase or solid phase (e.g., array) assays. Furthermore, the solid supports and oligomers can be used in probes of substantially any format, including, for example, format selected from molecular beacons, Scorpion Probes™, Sunrise Probes™, conformationally assisted probes, light up probes, Invader Detection probes, and TaqMan™ probes. See, for example, Cardullo, R., et al., Proc. Natl. Acad. Sci. USA, 85:8790-8794 (1988); Dexter, D. L., J. Chem. Physics, 21:836-850 (1953); Hochstrasser, R. A., et al., Biophysical Chemistry, 45:133-141 (1992); Selvin, P., Methods in Enzymology, 246:300-334 (1995); Steinberg, I., Ann. Rev. Biochem., 40:83-114 (1971); Stryer, L., Ann. Rev. Biochem., 47:819-846 (1978); Wang, G., et al., Tetrahedron Letters, 31:6493-6496 (1990); Wang, Y., et al., Anal. Chem., 67:1197-1203 (1995); Debouck, C., et al., in supplement to nature genetics, 21:48-50 (1999); Rehman, F. N., et al., Nucleic Acids Research, 27:649-655 (1999); Cooper, J. P., et al., Biochemistry, 29:9261-9268 (1990); Gibson, E. M., et al., Genome Methods, 6:995-1001 (1996); Hochstrasser, R. A., et al., Biophysical Chemistry, 45:133-141 (1992); Holland, P. M., et al., Proc Natl. Acad. Sci USA, 88:7276-7289 (1991); Lee, L. G., et al., Nucleic Acids Rsch., 21:3761-3766 (1993); Livak, K. J., et al., PCR Methods and Applications, Cold Spring Harbor Press (1995); Vamosi, G., et al., Biophysical Journal, 71:972-994 (1996); Wittwer, C. T., et al., Biotechniques, 22:176-181 (1997); Wittwer, C. T., et al., Biotechniques, 22:130-38 (1997); Giesendorf, B. A. J., et al., Clinical Chemistry, 44:482-486 (1998); Kostrikis, L. G., et al., Science, 279:1228-1229 (1998); Matsuo, T., Biochemica et Biophysica Acta, 1379:178-184 (1998); Piatek, A. S., et al., Nature Biotechnology, 16:359-363 (1998); Schofield, P., et al., Appl. Environ. Microbiology, 63:1143-1147 (1997); Tyagi S., et al., Nature Biotechnology, 16:49-53 (1998); Tyagi, S., et al., Nature Biotechnology, 14:303-308 (1996); Nazarenko, I. A., et al., Nucleic Acids Research, 25:2516-2521 (1997); Uehara, H., et al., Biotechniques, 26:552-558 (1999); D. Whitcombe, et al., Nature Biotechnology, 17:804-807 (1999); Lyamichev, V., et al., Nature Biotechnology, 17:292 (1999); Daubendiek, et al., Nature Biotechnology, 15:273-277 (1997); Lizardi, P. M., et al., Nature Genetics, 19:225-232 (1998); Walker, G., et al., Nucleic Acids Res., 20:1691-1696 (1992); Walker, G. T., et al., Clinical Chemistry, 42:9-13 (1996); and Compton, J., Nature, 350:91-92 (1991), the disclosures of which are each incorporated herein by reference in their entireties for all purposes.

In various embodiments, the present invention provides methods of detecting polymorphism in target nucleic acid sequences. Polymorphism refers to the occurrence of two or more genetically determined alternative sequences or alleles in a population. A polymorphic marker or site is the locus at which divergence occurs. Exemplary markers have at least two alleles, each occurring at frequency of greater than 1%, and more preferably greater than 10% or 20% of a selected population. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms.

In an exemplary embodiment, the solid support of the invention is utilized to detect a single nucleotide polymorphism. A single nucleotide polymorphism occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

In embodiments in which polymorphism is detected, polymorphic nucleic acids are bound to the solid support at addressable locations. Occurrence of a detectable signal at a particular location is indicative of the presence of a polymorphism in the target nucleic acid sequence.

In an exemplary embodiment, the probe is detectably labeled with a fluorophore moiety. There is a great deal of practical guidance available in the literature for selecting appropriate fluorophores for particular probes, as exemplified by the following references: Pesce et al., Eds., FLUORESCENCE SPECTROSCOPY (Marcel Dekker, New York, 1971); White et al., FLUORESCENCE ANALYSIS: A PRACTICAL APPROACH (Marcel Dekker, New York, 1970); and the like. The literature also includes references providing exhaustive lists of fluorescent and chromogenic molecules and their relevant optical properties for choosing fluorophores (see, for example, Berlman, HANDBOOK OF FLUORESCENCE SPECTRA OF AROMATIC MOLECULES, 2nd Edition (Academic Press, New York, 1971); Griffiths, COLOUR AND CONSTITUTION OF ORGANIC MOLECULES (Academic Press, New York, 1976); Bishop, Ed., INDICATORS (Pergamon Press, Oxford, 1972); Haugland, HANDBOOK OF FLUORESCENT PROBES AND RESEARCH CHEMICALS (Molecular Probes, Eugene, 1992) Pringsheim, FLUORESCENCE AND PHOSPHORESCENCE (Interscience Publishers, New York, 1949); and the like. Further, there is extensive guidance in the literature for derivatizing fluorophore molecules for covalent attachment via common reactive groups that can be added to a nucleic acid, as exemplified by the following references: Haugland (supra); Ullman et al., U.S. Pat. No. 3,996,345; Khanna et al., U.S. Pat. No. 4,351,760. Thus, it is well within the abilities of those of skill in the art to choose an energy exchange pair for a particular application and to conjugate the members of this pair to a probe molecule, such as, for example, a nucleic acid, peptide or other polymer.

In view of the well-developed body of literature concerning the conjugation of small molecules to nucleic acids, many other methods of attaching donor/acceptor pairs to nucleic acids will be apparent to those of skill in the art. For example, rhodamine and fluorescein dyes are conveniently attached to the 5′-hydroxyl of an nucleic acid at the conclusion of solid phase synthesis by way of dyes derivatized with a phosphoramidite moiety (see, for example, Woo et al., U.S. Pat. No. 5,231,191; and Hobbs, Jr., U.S. Pat. No. 4,997,928).

More specifically, there are many linker moieties and methodologies for attaching groups to the 5′- or 3′-termini of nucleic acids, as exemplified by the following references: Eckstein, editor, Nucleic acids and Analogues: A Practical Approach (IRL Press, Oxford, 1991); Zuckerman et al., Nucleic Acids Research, 15: 5305-5321 (1987) (3′-thiol group on nucleic acid); Sharma et al., Nucleic Acids Research, 19: 3019 (1991) (3′-sulfhydryl); Giusti et al., PCR Methods and Applications, 2: 223-227 (1993) and Fung et al., U.S. Pat. No. 4,757,141 (5′-phosphoamino group via Aminolink™ II available from P.E. Biosystems, CA.) Stabinsky, U.S. Pat. No. 4,739,044 (3-aminoalkylphosphoryl group); Agrawal et al., Tetrahedron Letters, 31: 1543-1546 (1990) (attachment via phosphoramidites linkages); Sproat et al., Nucleic Acids Research, 15: 4837 (1987) (5-mercapto group); Nelson et al., Nucleic Acids Research, 17: 7187-7194 (1989) (3′-amino group), and the like.

Means of detecting fluorescent labels are well known to those of skill in the art. Thus, for example, fluorescent labels can be detected by exciting the fluorophore with an appropriate wavelength of light and detecting the resulting fluorescence. The fluorescence can be detected visually, by means of photographic film, by the use of electronic detectors such as charge coupled solid supports (CCDs) or photomultipliers and the like. Similarly, enzymatic labels may be detected by providing the appropriate substrates for the enzyme and detecting the resulting reaction product.

Though exemplified by reference to detection of a fluorescent labeled nucleic acid, the solid supports of this invention are useful for the detection of analyte molecules. When the polymer is functionalized with a binding group, the solid support will capture onto the surface analytes that bind to the particular group. Unbound materials can be washed off, and the analyte can be detected in any number of ways including, for example, a gas phase ion spectrometry method, an optical method, an electrochemical method, atomic force microscopy and a radio frequency method. Exemplary optical methods include, for example, detection of fluorescence, luminescence, chemiluminescence, absorbance, reflectance, transmittance, birefringence or refractive index (e.g., surface plasmon resonance, ellipsometry, quartz crystal microbalance, a resonant mirror method, a grating coupler waveguide method (e.g., wavelength-interrogated optical sensor (“WIOS”) or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltammetry and amperometry methods. Radio frequency methods include multipolar resonance spectroscopy or interferometry. Optical methods include microscopy (both confocal and non-confocal), imaging methods and non-imaging methods. Immunoassays in various formats (e.g., ELISA) are popular methods for detection of analytes captured on a solid phase. Electrochemical methods include voltammetry and amperometry methods. Radio frequency methods include multipolar resonance spectroscopy.

Conditions that favor hybridization between an oligomer of the present invention and target nucleic acid molecules can be determined empirically by those skilled in the art, and can include optimal incubation temperatures, salt concentrations, length and base compositions of oligonucleotide analogue probes, and concentrations of oligomer and nucleic acid molecules of the sample. Preferably, hybridization is performed in the presence of at least one millimolar magnesium ion and at a pH that is above 6.0. In some embodiments, it may be necessary or desirable to treat a sample to render nucleic acid molecules in the sample single-stranded prior to hybridization. Examples of such treatments include, but are not limited to, treatment with base (preferably followed by neutralization), incubation at high temperature, or treatment with nucleases.

In addition, because the salt dependence of hybridization to nucleic acids is largely determined by the charge density of the backbone of a hybridizing oligonucleotide analogue, increasing the ratio of pPNA monomers in a HypNA-pPNA oligomer or a SerNA-pPNA oligomer of the present invention can increase the salt dependence of hybridization. This can be used to advantage in the methods of the present invention where it can in some aspects be desirable to be able to increase the stringency of hybridization by changing salt conditions, for example, or release a hybridized nucleic acid by reducing the salt concentration. In yet other aspects of the present invention, it can be desirable to have high-affinity binding of an oligonucleotide analogue of the present invention to a nucleic acid in very low salt. In this case, maintaining a ratio of close to 1:1 of HypNA to pPNA monomers in an oligonucleotide analogue of the present invention is advantageous.

The high degree of specificity of oligomers of the present invention in binding to target nucleic acid molecules allow the practitioner to select hybridization conditions that can favor discrimination between nucleic acid sequences that comprise a stretch of sequence that is completely complementary to at least a portion of one or more oligomer and target nucleic acid molecules that comprise a stretch of sequence that comprises a small number of non-complementary bases within a substantially complementary sequence. For example, hybridization or wash temperatures can be selected that permit stable hybrids between oligomer of the present invention and target nucleic acid molecules that are completely complementary along a stretch of sequence but promote dissociation of hybrids between oligomer of the present invention and target nucleic acid molecules that are not completely complementary, including those that comprise one or two base mismatches along a stretch of complementary sequence. The selection of a temperature for hybridization and washes can be dependent, at least in part, on other conditions, such as the salt concentration, the concentration of oligomer and target nucleic acid molecules, the relative proportions of oligomer to target nucleic acid molecules, the length of the oligomers to be hybridized, the base composition of the oligomer and target nucleic acid molecules, the monomer composition of the oligonucleotide analogue molecules, etc. In addition, when selecting for conditions that favor stable hybrids of completely complementary molecules and disfavor stable hybrids between oligomer and target nucleic acid molecules that are mismatched by one or more bases, additional conditions can be taken into account, and, where desirable, altered, including but not limited to, the length of the oligonucleotide analogue to be hybridized, the length of the stretch of sequence of complementarity between oligomer and target nucleic acid molecules, the number of non-complementary bases within a stretch of sequence of complementarity, the identity of mismatched bases, the identity of bases in the vicinity of the mismatched bases, and the relative position of any mismatched bases along a stretch of complementarity. Those skilled in the art of nucleic acid hybridization would be able to determine favorable hybridization and wash conditions in using oligomers of the present invention for hybridization to target nucleic acid molecules, depending on the particular application. “Favorable conditions” can be those favoring stable hybrids between oligomer and target nucleic acid molecules that are, at least in part, substantially complementary, including those that comprise one or more mismatches.

“Favorable conditions” can be those favoring stable hybrids between oligomer and target nucleic acid molecules that are, at least in part, completely complementary and disfavor or destabilize hybrids between molecules that are not completely complementary.

Using methods such as those disclosed herein, the melting temperature of oligomer of the present invention hybridized to target nucleic acid molecules of different sequences can be determined and can be used in determining favorable conditions for a given application. It is also possible to empirically determine favorable hybridization conditions by, for example, hybridizing target nucleic acid molecules to oligomer that are attached to a solid support and detecting hybridized complexes.

Target nucleic acid molecules that are bound to solid supports or oligomeric probes of the present invention can be conveniently and efficiently separated from unbound nucleic acid molecules of the survey population by the direct or indirect attachment of oligomer probes to a solid support. A solid support can be washed at high stringency to remove nucleic acid molecules that are not bound to oligomer probes. However, the attachment of oligomer probes to a solid support is not a requirement of the present invention. For example, in some applications bound and unbound nucleic acid molecules can be separated by centrifugation through a matrix or by phase separation or some by other forms of separation (for example, differential precipitation) that can optionally be aided by chemical groups incorporated into the oligomer probes (see, for example, U.S. Pat. No. 6,060,242 issued May 9, 2000, to Nie et al.).

In an exemplary embodiment, a solid support of the invention is utilized in a real time PCR assay such as those described in commonly owned, copending U.S. patent application Ser. No. 13/399,872.

In other methods of the invention, the present invention is directed to a method for preparing a solid support having a probe molecule bound thereto, the method comprising contacting the solid support comprising an azide or alkyne moiety covalently bound to the outer surface of the solid support (as described herein above) with the polymer comprising A, B and C subunits described herein above.

In other embodiments, the methods further comprise contacting a Cu(I) catalyst with the solid support and the polymer. Further embodiments comprise contacting a probe molecule having an amine functional group with the solid support comprising a polymer bound thereto to prepare a solid support comprising a probe molecule bound thereto. Such methods have utility in any number of applications, such as preparation of DNA microarrays and the like.

The following examples are provided for purposes of illustration, not limitation.

### EXAMPLES

### Example 1

### Preparation of poly(N,N-dimethylacrylamide-co-pentafluorophenyl Acrylate), Poly(DMA-co-PFPA) at a Molar Feed Ratio of 35% DMA—A General Procedure

A solution of 2.24 g (22.58 mmol, 35 mol %) of dimethylacrylamide (DMA), 10.01 g (42.03 mmol, 65 mol %) of pentafluorophenyl acrylate (PFPA) and 10.1 mg (0.041 mmol) of 2,2′-Azobis(2,4-dimethylvaleronitrile) in 30 mL of anhydrous acetonitrile in a 150-mL round-bottom glass flask was purged (bubbling) with ultra pure argon at a flow rate of about 60 mL/min and magnetic stirring at 200 rpm for 45 minutes. The reaction flask was then lowered into an oil bath at 55° C. The argon flow rate and magnetic stirring were reduced to about 25 mL/min and 120 rpm, respectively. The polymerization was conducted under such conditions for 19 hours. The viscous reaction mixture was cooled down to ambient temperature and exposed to ambient atmosphere prior to workup.

The acetonitrile was removed under reduced pressure (Rotavap) at ˜55° C. in a water bath for 30 minutes, and the residual monomers were removed in a vacuum oven at 0.5 millibar and 59° C. for 3 hours. The polymer product was re-dissolved in 40 mL of anhydrous THF while stirring in an oil bath at 55° C. open air. With magnetic stirring, about 50 mL of n-hexane was added dropwise until the solution turned slightly cloudy. To 1400-mL of n-hexane in a 2-L poly(propylene) Erlenmeyer flask, continuously flooded with dry nitrogen, the cloudy suspension was added through a 22-gauge syringe needle in a fine stream while stirred vigorously using a 2″ PTFE stirring blade. The precipitated polymer was stirred for an additional 5 minutes and then transferred into 600 mL of fresh n-hexane with gentle stirring for an additional 5 minutes. The polymer was transferred into another 600 mL of fresh n-hexane and soaked for 15 minutes. The precipitated polymer was in the shape of coarse fibers. It was transferred into a large mouth 500-mL glass bottle and dried under vacuum at 55° C. for 22 hours to give 10.6792 g (87.1% yield) of poly(DMA-co-PFPA). By quantitative ¹⁹F-NMR the molar incorporation percentage of PFPA was found to be 67% (by inference DMA 33%) and the extent of ester hydrolysis to be 1.4%.

This general procedure for polymerization is applicable to the preparation of homopolymer, copolymers and terpolymers having any desired ratios of diluent and reactive monomer.

### Example 2

### Preparation of poly(N,N-dimethylacrylamide-co-pentafluorophenyl Acrylate), poly(DMA-co-PFPA) at a Molar Feed Ratio of 39% DMA

The above general procedure was followed. A solution of 2.5816 g (26.043 mmol, 39 mol %) of DMA, 9.7003 g (40.740 mmol, 61 mol %) of PFPA and 10.6 mg (0.043 mmol) of 2,2′-Azobis(2,4-dimethylvaleronitrile) in 30 mL of anhydrous acetonitrile in a 150-mL round-bottom glass flask was purged (bubbling) with ultra pure argon at a flow rate of about 60 mL/min and magnetic stirring at 200 rpm for 45 minutes. The reaction flask was then lowered into an oil bath at 55° C. The argon flow rate and magnetic stirring were reduced to about 25 mL/min and 120 rpm, respectively. The polymerization was conducted under such conditions for 19 hours to give 10.42 g (84.8% yield) of poly(DMA-co-PFPA). By quantitative ¹⁹F-NMR the molar incorporation percentage of PFPA was found to be 58% (by inference DMA 42%) and ester hydrolysis to be 1.9%.

### Example 3

### Preparation of poly(N,N-dimethylacrylamide-co-pentafluorophenyl Acrylate), poly(DMA-co-PFPA) at a Molar Feed Ratio of 15% DMA

The general procedure was followed. A solution of 0.6090 g (6.143 mmol, 15 mol %) of DMA, 8.2395 g (34.605 mmol, 85 mol %) of PFPA and 10.6 mg (0.043 mmol) of 2,2′-Azobis(2,4-dimethylvaleronitrile) in 30 mL of anhydrous acetonitrile in a 150-mL round-bottom glass flask was purged (bubbling) with ultra pure argon at a flow rate of about 60 mL/min and magnetic stirring at 200 rpm for 45 minutes. The reaction flask was then lowered into an oil bath at 55° C. The argon flow rate and magnetic stirring were reduced to about 25 mL/min and 120 rpm, respectively. The polymerization was conducted under such conditions for 6 hours to give 7.908 g (89.4% yield) of poly(DMA-co-PFPA). By quantitative ¹⁹F-NMR the molar incorporation percentage of PFPA was found to be 87% (by inference DMA 13%) and the ester hydrolysis to be 1.7%.

### Example 4

### Preparation of poly(N,N-dimethylacrylamide-co-acrylamide-co-pentafluorophenyl Acrylate), poly(DMA-co-AAm-co-PFPA), at a Molar Feed Ratio of 34% DMA

The general procedure was followed. A solution of 2.1500 g (21.689 mmol, 34 mol %) of DMA, 2304 g (3.241 mmol, 5 mol %) of AAm, 9.2504 g (38.851 mmol, 61 mol %) of PFPA and 9.9 mg (0.040 mmol) of 2,2′-Azobis(2,4-dimethylvaleronitrile) in 30 mL of anhydrous acetonitrile in a 150-mL round-bottom glass flask was purged (bubbling) with ultra pure argon at a flow rate of about 60 mL/min and magnetic stirring at 200 rpm for 45 minutes. The reaction flask was then lowered into an oil bath at 55° C. The argon flow rate and magnetic stirring were reduced to about 25 mL/min and 120 rpm, respectively. The polymerization was conducted under such conditions for 23 hours to give 10.351 g (88.9% yield) of terpoly(DMA-co-AAm-co-PFPA). By quantitative ¹⁹F-NMR the molar incorporation percentage of PFPA was found to be 59% (by inference DMA+acrylamide 41%) and ester hydrolysis to be 3.5%.

### Example 5

### Preparation of poly(acrylamide-co-pentafluorophenyl Acrylate, poly(AAm-co-PFPA), at a Molar Feed Ratio of 15% AAm

The general procedure was followed. A solution of 0.2123 g (2.987 mmol, 14.8 mol %) of acrylamide (AAm), 4.0921 g (17.186 mmol, 85.2 mol %) of PFPA and 4.7 mg (0.019 mmol) of 2,2′-Azobis(2,4-dimethylvaleronitrile) in 20 mL of anhydrous acetonitrile in a 150-mL round-bottom glass flask was purged (bubbling) with ultra pure argon at a flow rate of about 60 mL/min and magnetic stirring at 200 rpm for 45 minutes. The reaction flask was then lowered into an oil bath at 55° C. The argon flow rate and magnetic stirring were reduced to about 25 mL/min and 120 rpm, respectively. The polymerization was conducted under such conditions for 19 hours. The solvent and residual PFPA were removed under reduced pressure (Rotavap) at ˜55° C. water bath temperature for 30 minutes, and in a vacuum oven at 0.5 millibar and 55° C. for 3 hours. The polymer product was re-dissolved in 20 mL of anhydrous THF while stirred constantly with a magnetic stir bar in an oil bath at 55° C. With constant stirring, 45 mL of n-hexane was added dropwise to give a slightly cloudy solution. To 1200 mL of n-hexane in a 2-L glass Erlenmeyer flask, continuously flooded with dry nitrogen, the cloudy solution was added through a 22-gauge syringe needle in a fine stream while stirred vigorously using a 2″ PTFE stirring blade. The precipitated polymer was suction-filtered, rinsed with plenty of n-hexane (˜400 mL), suction air-dried, and vacuum dried at 55° C. overnight to give 3.24 g (75.1% yield) of poly(AAm-co-PFPA). The copolymer is soluble in acetonitrile, acetone, THF and chloroform.

### Example 6

### Preparation of poly(pentafluorophenyl acrylate)(poly(PFPA))

The general procedure was followed. A solution of 12.0033 g (50.413 mmol) of PFPA and 9.9 mg (0.040 mmol) of 2,2′-Azobis(2,4-dimethylvaleronitrile) in 30 mL of anhydrous acetonitrile in a 150-mL round-bottom glass flask was purged (bubbling) with ultra pure argon at a flow rate of about 60 mL/min and magnetic stirring at 200 rpm for 45 minutes. The reaction flask was then lowered into an oil bath at 55° C. The argon flow rate and magnetic stirring were reduced to about 25 mL/min and 120 rpm, respectively. The polymerization was conducted under such conditions for 18 hours.

At the end of 18 hours, the solvent and residual monomer were removed under reduced pressure (Rotavap) at ˜55° C. in a water bath for 30 minutes, and under high vacuum at 55° C. for 5 hours. The polymer product was re-dissolved in 30 mL of anhydrous THF while stirring in an oil bath at 55° C. open air. With magnetic stirring, about 15 mL of n-hexane was added dropwise until the solution turned slightly cloudy. To 1200-mL of n-hexane in a 2-L poly(propylene) Erlenmeyer flask, continuously flooded with dry nitrogen, the cloudy suspension was added through a 22-gauge syringe needle in a fine stream while stirred vigorously using a 2″ PTFE stirring blade. The precipitated polymer was transferred into 500 mL of fresh n-hexane and stirred for an additional 10 minutes. The polymer was then transferred into another 500 mL of fresh n-hexane and soaked for 15 minutes. The precipitated polymer was in the shape of coarse fibers. It was transferred into a large mouth 500-mL glass bottle and dried under vacuum at 55° C. for 22 hours to give 9.96 g (83.0% yield) of poly(PFPA). The homopolymer is insoluble in water, slightly soluble in acetonitrile, but readily soluble in THF, acetone and chloroform. By quantitative ¹⁹F-NMR the molar incorporation percentage of PFPA was found to be 100.9% of the calculated value (within NMR sensitivity range) and ester hydrolysis undetectable.

### Example 7

### Covalent Immobilization of a Polymer onto a Plastic Substrate Surface—A General Procedure

Atmospheric Pressure Oxygen Plasma generator, ATOMFLO™ Model 400 equipped with a 1″ linear plasma source (Surfx Technologies, Culver, Calif.), and an X-Y Robot, F4200N, (Fisnar, Wayne, N.J.) are used to introduce oxygenated functional groups onto plastic substrate surfaces. Plastic samples are placed on the aluminum scanning platform of the robot having the surfaces to be treated facing up to the plasma source 4 mm above. The plasma is generated at 60 W with helium and oxygen flow rates of 15 L/min and 0.05 L/min, respectively. The plasma source scans across the substrate surfaces at a speed of 20 mm/sec. The number of scanning varies from 1 to 10 times in order to tailor the surface densities of hydroxyl, carbonyl, and carboxylic functional groups.

The plasma-treated substrate samples are immersed in an acetonitrile or acetone solution of a coating polymer and a base catalyst, and tumbled gently at ambient temperature for 2 to 20 hours. The substrate samples are removed, rinsed with plenty of acetonitrile or acetone, and blow-dried with nitrogen.

### Example 8

### Covalent Immobilization of Poly(DMA-co-PFPA), 35:65 mol % of DMA:PFPA on Cop Substrate Chips

The general procedure for covalent immobilization of a polymer is followed with 3 passes of plasma scanning Four plasma-treated COP substrate samples (chips), 67 mm×25 mm×1.0 mm, are immersed in 25 mL of anhydrous acetonitrile solution containing 15.0 μm of triethylamine and 87.3 mg of 35:65 mol % DMA:PFPA copolymer. After gentle tumbling for 19 hours, the COP substrate chips are removed, rinsed with plenty of acetonitrile, and blow-dried. The polymer-immobilized surfaces exhibit water contact angles of 69.4±0.8 degrees (n=9).

Under similar conditions, 4 plasma-treated COP substrate chips are immersed in 25 mL of anhydrous acetonitrile solution containing 16.2 μL of N,N-dimethylbenzylamine, and 87.3 mg of 35:65 mol % DMA:PFPA copolymer. After rinsing with acetonitrile and blow-drying, the polymer-immobilized surfaces exhibit water contact angles of 89.5±0.6 degrees (n=8).

### Example 9

### Covalent Immobilization of poly(PFPA) on COP Substrate Chips

The general procedure for covalent immobilization of a polymer is followed with 3 passes of plasma scanning Four plasma-treated COP substrate samples (chips), 67 mm×25 mm×1.0 mm, are immersed in 25 mL of acetone solution containing 15.0 μL of triethylamine and 86.7 mg of PFPA homopolymer. Other solvents, such as acetonitrile, may also be used. After gentle tumbling for 17 hours, the COP substrate chips are removed, rinsed with plenty of acetone, and blow-dried. The polymer-immobilized surfaces exhibit water contact angles of 78.8±0.8 degrees (n=10).

Under similar conditions, 4 plasma-treated COP substrate chips are immersed in 25 mL of acetone solution containing 16.2 μL of N,N-dimethylbenzylamine, and 86.7 mg of PFPA homopolymer. After rinsing with acetone and blow-drying, the polymer-immobilized surfaces exhibit contact angles of 86.7±0.5 degrees (n=6).

Under similar conditions, 12 COP substrate chips are pre-treated by plasma with only one pass. These pre-treated COP substrate chips, 4 in a set, are immersed in 25 mL of acetone solution containing 16.2 μL of N,N-dimethylbenzylamine and 86.0 mg of PFPA homopolymer and tumbled at ambient temperature for 16 hours. After rinsing with acetone and blow-drying, the polymer-immobilized surfaces exhibit water contact angles are 83.6±0.4 degrees (n=18).

### Example 10

### Covalent Immobilization of terpoly(Acrylamide-co-DMA-co-PFPA), 5:34:61 mol % of AAm:DMA:PFPA, on COP Substrate Chips

The general procedure for covalent immobilization of a polymer is followed with 3 passes of plasma scanning Four plasma-treated COP substrate samples (chips), 67 mm×25 mm×1.0 mm, are immersed in 25 mL of anhydrous acetonitrile solution containing 15.0 μL of triethylamine and 87.3 mg of terpoly(AAm-co-DMA-co-PFPA). Other solvents, such as acetonitrile, may also be used. After gentle tumbling for 17 hours, the COP substrate chips are removed, rinsed with plenty of acetonitrile, and blow-dried. The polymer-immobilized surfaces exhibit water contact angles of 76.7±0.6 degrees (n=7).

Under similar conditions, 4 plasma-treated COP substrate chips are immersed in 25 mL of anhydrous acetonitrile solution containing 16.2 μL of N,N-dimethylbenzylamine, and 87.3 mg of terpoly(AAm-co-DMA-co-PFPA) to give polymer-immobilized surfaces having water contact angles of 77.5±0.2 degrees (n=6).

### Example 11

### Covalent Immobilization of poly(Acrylamide-co-PFPA), 15:65 mol % of AAm:PFPA, on COP Substrate Chips

The general procedure for covalent immobilization of a polymer is followed with one pass of plasma scanning Four plasma-treated COP substrate samples (chips), 67 mm×25 mm×1.0 mm, are immersed in 25 mL of acetone solution containing 16.2 μL of triethylamine and 85.8 mg of ploy(AAm-co-PFPA). Other solvents, such as acetonitrile, may also be used. After gentle tumbling for 20 hours, the COP substrate chips are removed, rinsed with plenty of acetone, and blow-dried. The polymer-immobilized surfaces exhibit water contact angles of 82.5±0.5 degrees (n=18).

### Example 12

### Ammonia Capping Procedure for Reactive Polymer-Coated Solid Supports

### Procedure:

An aqueous solution was prepared containing 50 mM ammonium hydroxide and 100 mM triethylamine. A portion of the solution (25 mL) was poured into a 30 mL screw-top polypropylene slide tube containing 4 pieces of polymer support slides, 1″×3″×0.04″ polymer-coated COP (cyclic olefin polymer) slides. The slides were prepared as described above by covalently binding a reactive polymer to hydroxyl groups on the substrate surface (to form an ester linkage) and were previously spotted with capture probe microarrays but not washed. The tube was sealed and placed in a water bath at 60° C. for 1 hr, after which time the ammonia solution was decanted and replaced with water. After a minute, slides were removed, rinsed with additional water, and blown dry under a nitrogen stream. An average water contact angle (WCA) measurement of about 8 degrees was obtained for the capped slide surfaces. The average water contact angle (WCA) for the polymer-coated slides support prior to capping was 86 degrees. Each water contact angle was taken as the average of three measurements on each of 4 pieces of solid support slides.

### Optimization of Ammonia Capping:

To determine the parameters for capping additional, solutions were prepared containing either 100 mM or 500 mM ammonium hydroxide, each containing 100 mM triethylamine. Solid support slides were capped in each of these solutions and in a tube containing water alone for 1 hr at 4 different temperatures, 20° C., 60° C., 75° C., and 95° C. Table 1 presents data for solid support slides prepared with a copolymer having 65 mol % PFPA and 35 mol % DMA over a range of reagent concentrations and at four immersion temperatures for 1 hr.

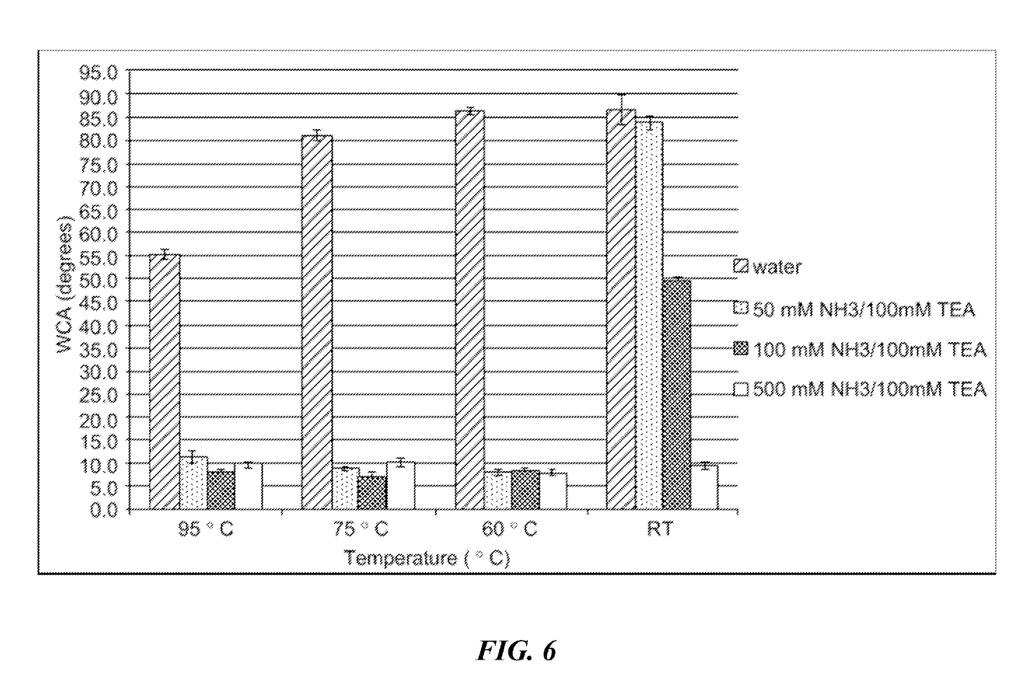

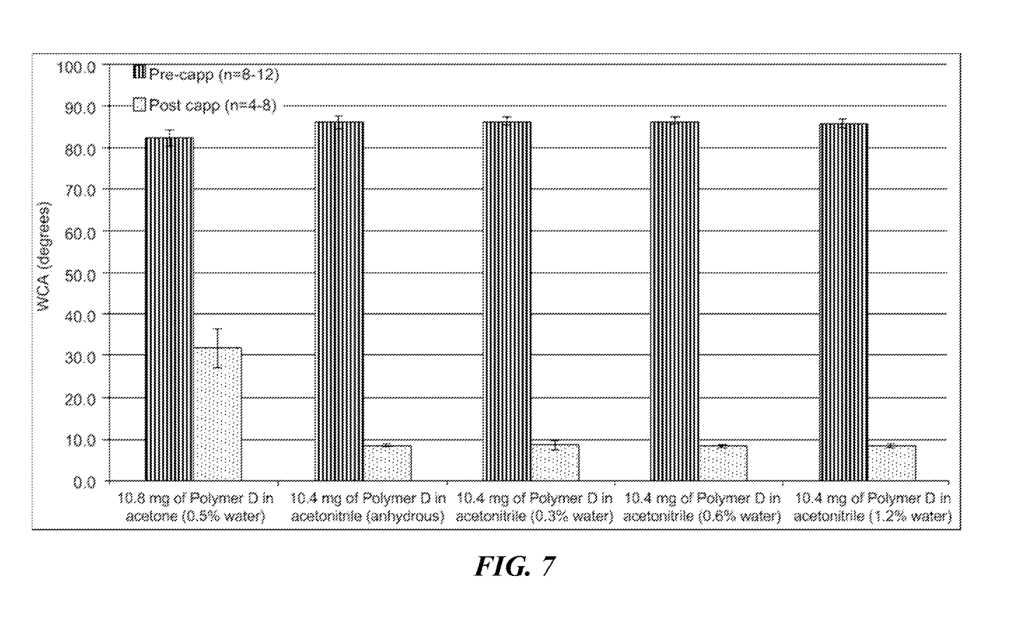

The water contact angle prior to capping was 86 degrees. A negligible difference in final water contact angle was obtained (8 degrees to 10 degrees) with 500 mM ammonia at any temperature. At 20° C. a reagent concentration dependence is observed, where 50 mM ammonia affects little change in water contact angle, 500 mM affects the maximum change (minimum water contact angle), and 100 mM produces an intermediate water contact angle (50 degrees). At temperatures 60° C. and above the maximum water contact angle change is obtained independent of ammonia concentration. Water treatment alone does not significantly change water contact angle except at the highest temperature (T=95° C., water contact angle=55 degrees). Each water contact angle listed in Table 1 is the average of 3 individual measurements on each of four solid support slides. The data from Table 1 (shown graphically in FIG. 6) illustrates the effect on final WCA of ammonia capping using COP slides immobilized with poly(PFPA-co-DMA), 67.5% PFPA and 32.5% DMA, over a range of reagent concentrations and immersion temperatures. WCA prior to capping was 86°.

TABLE 1

| | WCA | | | | StdDev | | | |
|---|---|---|---|---|---|---|---|---|
| | Temp ° C. | | | | | | | |
| Reagent | 95 | 75 | 60 | RT | 95 | 75 | 60 | RT |
| water | 55.1 | 81.0 | 86.3 | 86.7 | 1.0 | 1.1 | 0.8 | 3.2 |
| 50 mM NH₃/ | 11.4 | 8.9 | 8.0 | 83.7 | 1.3 | 0.4 | 0.7 | 1.5 |
| 100 mM TEA | | | | | | | | |
| 100 mM NH₃/ | 8.3 | 7.3 | 8.6 | 49.4 | 0.2 | 0.8 | 0.3 | 0.9 |
| 100 mM TEA | | | | | | | | |
| 500 mM NH₃/ | 9.7 | 10.3 | 8.0 | 9.5 | 0.7 | 0.8 | 0.6 | 0.8 |
| 100 mM TEA | | | | | | | | |

The WCA of the present solid supports after capping is significantly lower than the WCA of currently known solid supports after capping. While not wishing to be bound by theory, it is believed that the lower WCA of the present solid supports is related, at least in part, to the stability of the covalent linkage (W) under capping conditions. Currently available solid supports comprise different, less stable linkages (e.g., formed by UV activation) and capping of such supports is believed to lead to cleavage of polymer from the substrate, and thus an increase in WCA (due to more exposed substrate surface area). As noted above, the decrease in WCA associated with the present solid supports is advantageous in many respects, including dissolution of PCR and/or other analytical reagents used in combination with the solid supports.

### Example 13

### NMR Analysis of the Polymers

While the feed ratio of monomers in a copolymer or terpolymer are known by weight or volume measurement, the actual ratio of monomers incorporated into the product is variable and must be determined post-synthesis. Each batch of polymer is characterized by ¹H, ¹³C, and quantitative ¹⁹F NMR; each NMR method provides key information about the final product. Experimental parameters, examples of spectra, and information obtained are discussed below. Pentafluorophenyl acrylate-containing polymers are generally quite soluble in chloroform-d (CDCl₃) and this solvent is generally preferred as chemical shifts are predictable and room-temperature line width optimal.

### ¹H-NMR

Proton spectra are collected at 400 MHz. Copolymers of pentafluorophenyl acrylate with dimethylacrylamide (or acrylamide) are typified by broad peaks and cannot be assigned due to overlapping signals in the δ1-4 ppm region where the polymer backbone and amide signals occur. Furthermore, the fluorinated monomer possesses no protons on the aryl ring and thus contributes only to the backbone signal. However, proton spectra are useful as they reveal the presence of unreacted monomers, if any are present, as sharp peaks in the δ5-7 ppm region. Water in the sample may be observed as a sharp peak in chloroform at δ1.6 ppm. Contamination of the polymer by traces of processing solvents, such as hexane, may also be observed as sharp signals. All the signals arising from contaminants may be integrated to estimate overall purity of the copolymer. Acceptable polymers will contain less than 0.5 molar percent total monomer content. Traces of solvents such as hexane are of little concern other than in correctly estimating the concentration of polymer during subsequent use.

### ¹³C-NMR

Carbon spectra are acquired at 100 MHz, proton decoupled, with a sweep width of 25K, pulse width of 4.4 μsec at 30 degrees, and 1.5 sec pulse delay. A typical sample of 50 mg polymer in 500 uL solvent will require 16K scans, allowing semi-quantitative observation of the carbonyl carbons (amide and ester) from each of the monomers (δ165-175 ppm). Unlike proton spectra, ¹³C line width of polymers is also narrow enough to allow assignment of the three types of fluorinated carbons and to differentiate methyl peaks on the amide from backbone carbons.

### ¹⁹F-NMR

Fluorine spectra are collected at 376 MHz, non-proton decoupled, sweep width 90K, and pulse width 7.8 μsec at 45 degrees. For quantitative analysis 32 scans with a pulse delay of 60 sec are required. A typical sample consists of 20-30 mg polymer in 500 uL CDCl₃ containing 2-3 mg of fluorobenzene as internal standard. Based on the feed ratio, a unit average monomeric FW (Mc) is calculated by Mc=a(Mₚ)+b(M_{d}), where Mp is the FW of the reactive comonomer fragment (238.11 for PFPA), M_{d} is the FW of the diluent comonomer fragment (99.13 for DMA), and a and b are the mol fractions in the polymerization solution (a+b=1). Based on the calculated unit FW for a particular PFPA feed ratio and the known weight of a sample (and weight of added fluorobenzene) the actual fluorine incorporated into the polymer may be determined. Where the polymer is a terpolymer containing acrylamide as well as dimethylacrylamide, a small unit FW correction factor is applied; however, only the PFPA incorporation percentage can be deduced by NMR. The fluorine spectra are also useful in observing any ester hydrolysis, as the free pentafluorophenol resonances are usually sharp and well-separated from the polymeric fluorine signal, thus allowing quantitative assessment of remaining active ester content. For PFPA polymers the ¹⁹F signals occur around δ-152 (2F), -157 (1F), and -162 (2F) ppm as wide but easily integrated peaks, while the pentafluorophenol peaks appear around δ-161 (2F), -165 (2F), and -171 (1F) ppm as sharp multiplets. The internal fluorobenzene standard appears as narrow complex multiplet at around δ-112 ppm.

### Exemplary Analysis

As an example, a PFPA-DMA copolymer with a molar feed ratio 85:15 gave a total integrated fluorine signal corresponding to 91.7 μmol PFP groups (based on the addition of a known amount of fluorobenzene). For a copolymer of this composition the average monomeric FW=217.5, and a sample of 23.2 mg represents 106.7 μmol of monomeric units. Dividing the mol PFP by the calculated unit FW, (91.7/106.7)=0.86, or 86% PFPA incorporated. However, if the assumption were made that the copolymer composition is 87% PFPA, the slightly higher average monomeric unit FW gives an incorporation percent exactly matching the NMR observed fluorine content, i.e. (91.68/105.5)=87% PFPA; thus the actual incorporation rate is 87% PFPA.

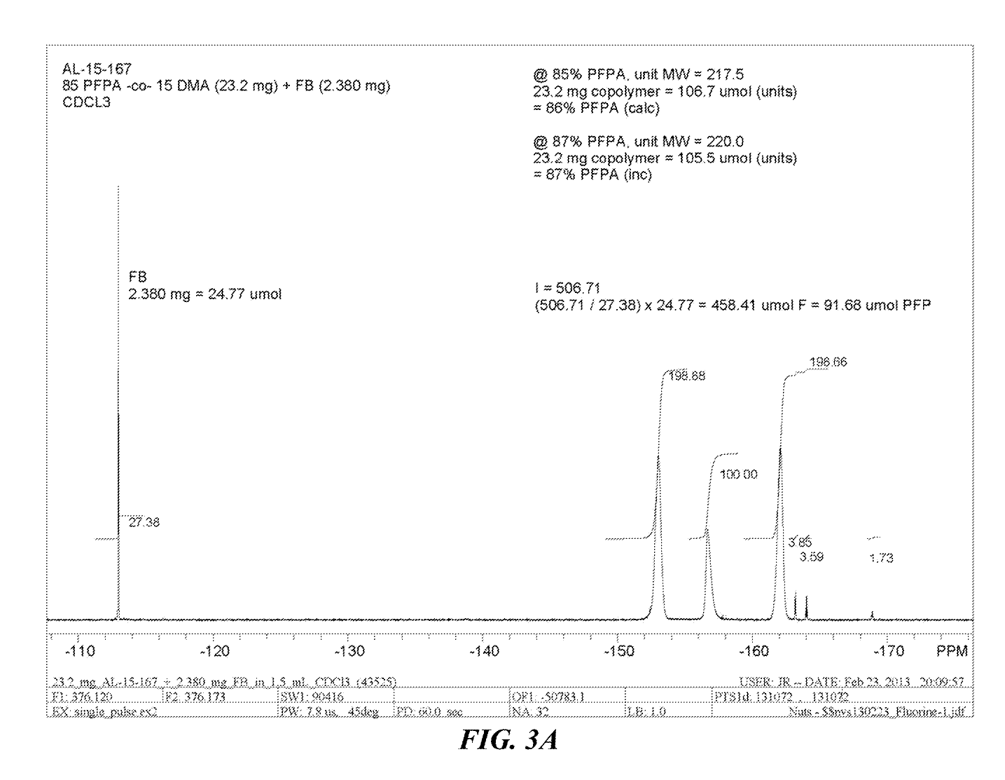

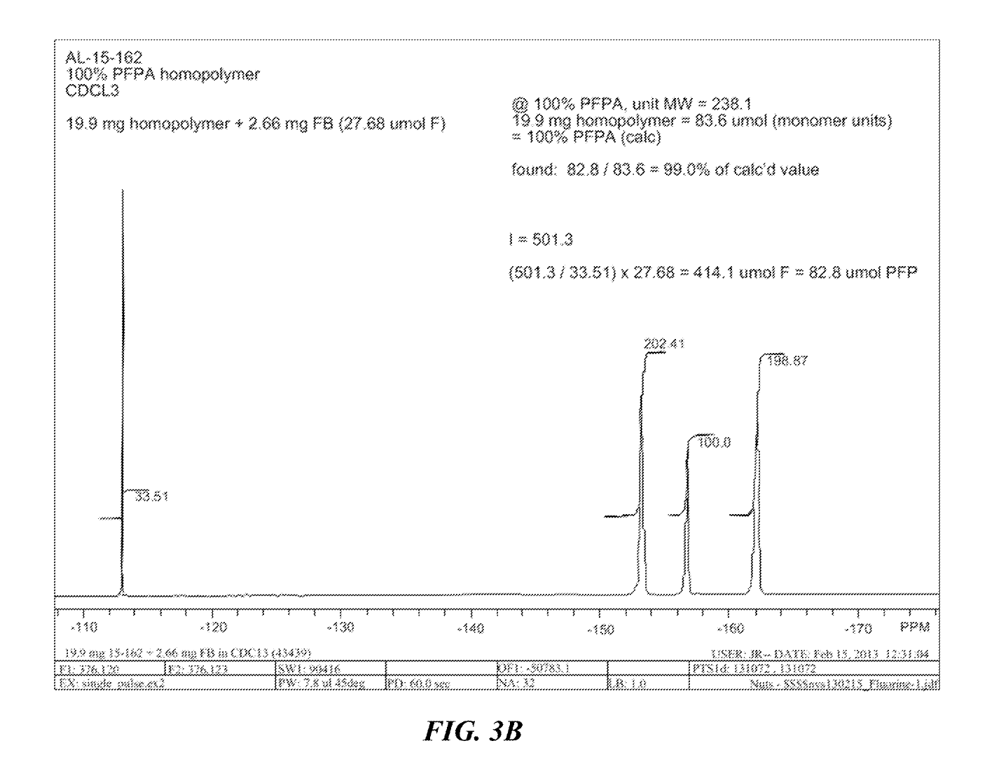

The ¹⁹F NMR for this example is presented in FIG. 3.

### Example 14

### Preparation of Oligonucleotide Arrays and Devices

Spotting solutions of 20 μM amine-modified oligonucleotides in 50 mM sodium phosphate (pH 8.5) are prepared in a 384-well plate. Oligos are then spotted onto a solid support prepared above in the desired pattern by an array spotter (Array-it SpotBot3), with an appropriate spotting pin selected for the desired spot size. Two arrays are spotted per slide at points ¼ and ¾ of the slide length, and centered in relation to the slide width. Following spotting, the slides are incubated at 75% relative humidity for 4-18 hours, then rinsed with a stream of DI water and blown dry with argon.

Following drying, slides are cut in half, resulting in two 1″×1.5″ chips with the spotted array centered on each. A small single-chamber device is assembled in which the spotted slide formed the bottom. A pre-cut double-sided PSA gasket of appropriate dimensions is placed on the slide, leaving the array-spotted portion exposed along with a roughly circular area of fixed dimension around it. On top of this gasket, a polycarbonate lid with two pre-drilled filling ports is placed. The resulting assembly is laminated at room temperature in order to insure proper adhesion during thermocycling.

Multiplex PCR solutions comprising primer and probe mix, buffer, enzyme, and target DNA are premixed in a tube and then added to the chamber described above. Typical reaction chamber volumes are 25-40 μL. Following addition of the PCR reaction solution the ports in the ports in the polycarbonate lid of the chip are sealed with an optically clear film.

Devices filled with PCR reaction solutions are tested in a custom thermocycling apparatus, which allows for imaging of the surface with a digital camera though an epifluoresence microscope during the course of thermocycling. Typical hybridization times for cleaved fluorescent DNA-flaps (and for full probes) is less than 2 minutes when cooled below their hybridization temperatures (Tₘ). Surfaces are characterized by measuring the fluorescence intensity of the cleaved flaps (or full probes) that hybridize to the capture probe array. In this manner, surface stability is measured in buffer under typical thermocycling conditions. PCR in the device is also conducted, with a run typically comprising activation at 95° C. for the desired time, 40 cycles of thermocycling from 95° C. to 60° C., with 15 sec. dwell time at 95° C. and 60 sec. dwell time at 60° C. At certain, chosen cycles, the chamber is chilled below the Tₘ of the probes, allowing for hybridization following the 60° C. extension step.

Automated image analysis software is utilized to locate the arrayed spots and to quantitate the signal by measuring pixel intensity. The average pixel intensity outside the actual spot area is subtracted from the average pixel intensity inside the spot, resulting in a background-subtracted pixel intensity for the spot regions. These intensities are monitored over the course of thermocycling for the detection of cleaved DNA-flaps specific to the capture probes.

### Example 15

### Thermocycling of a Solid Support

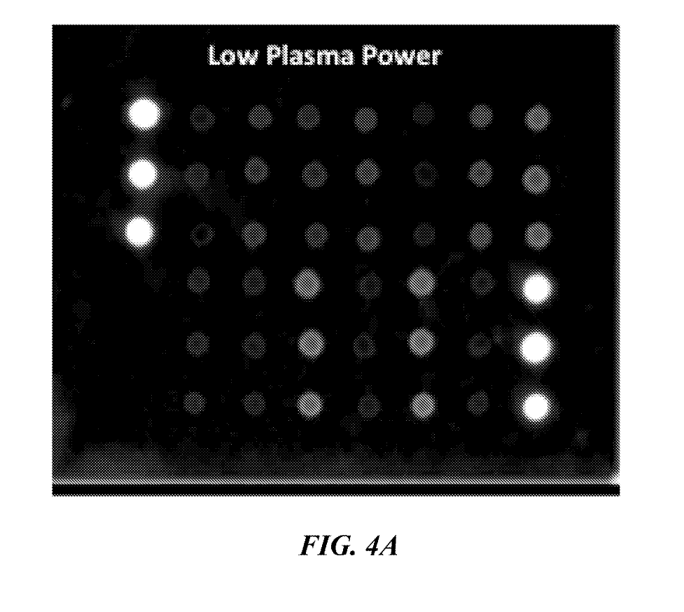

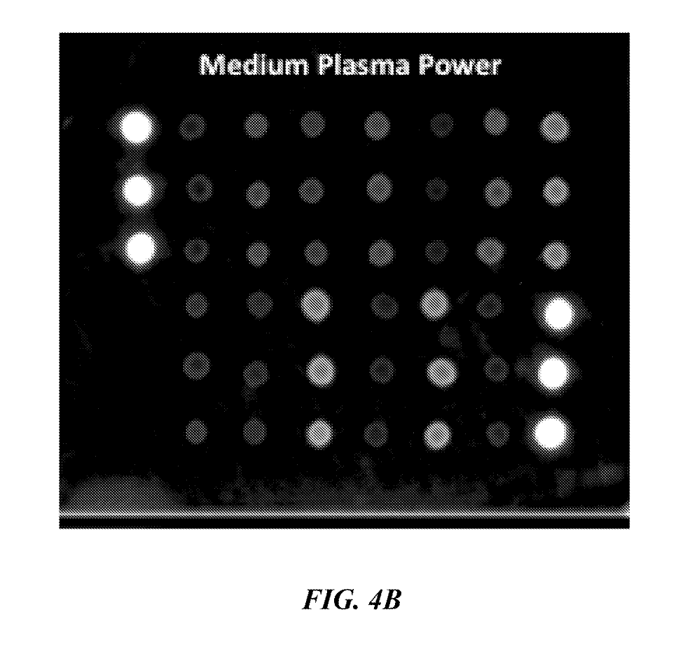

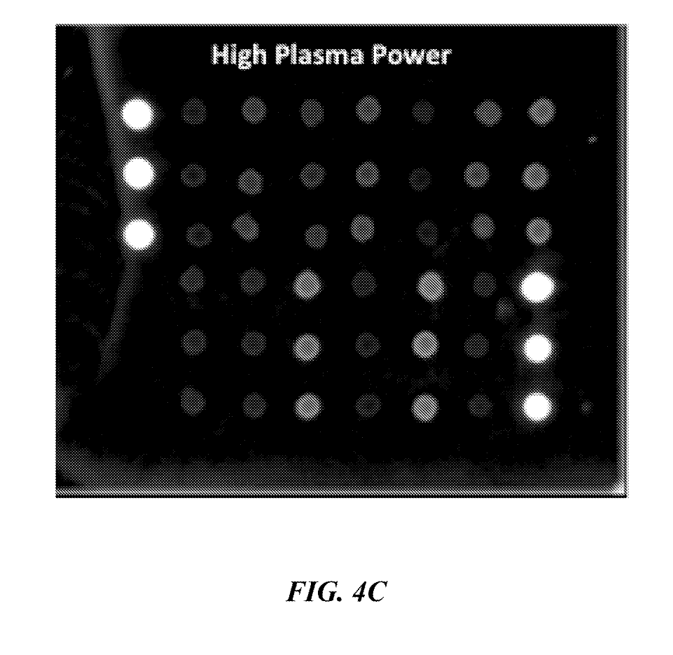

Cyclic poly(olefin) (COP) slides comprising poly(DMA-co-PFPA) polymers covalently bound thereto were prepared according to the procedures above. These solid supports were used to fabricate microarrays by spotting labeled oligos onto the solid supports. These microarrays were subjected to thermal cycling for 40 cycles (64 to 95° C.) in the presence of a buffer, and the spot shape and brightness were monitored. FIGS. 4A-C show results of arrays prepared with low, medium and high plasma power treated slide, respectively. As seen in FIGS. 4A-C, the spots remained intact after 40 cycles, indicative of covalent attachment, instead of non-specific adsorption, of the poly(DMA-co-PFPA) polymer onto the plasma (APOP)-treated COP slide.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including but not limited to U.S. Patent Application No. 61/785,987, filed Mar. 14, 2013, are incorporated herein by reference, in their entirety. Aspects of the embodiments can be modified, if necessary to employ concepts of the various patents, applications and publications to provide yet further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A solid support comprising:
a substrate having an outer surface; and
a plurality of polymers covalently bound to the outer surface of the substrate, the polymers each comprising at least one A and C subunit and optionally comprising one or more B subunits, wherein:
the A subunit, at each occurrence, independently comprises:
a) a first thermochemically reactive group, wherein the first thermochemically reactive group is capable of forming a covalent bond with an alcohol, carbonyl or amine group on a capture probe;
b) a second thermochemically reactive group, wherein the second thermochemically reactive group is a cycloaddition or conjugate addition reactive group having a reactivity specific for covalent bond formation with a target functional group on a capture probe via a cycloaddition or 1,4-conjugate addition reaction; or
c) a covalent bond to a capture probe,
the optional B subunit, at each occurrence, independently comprises a hydrophilic moiety; and
the C subunit, at each occurrence, independently comprises a covalent attachment W to the outer surface of the substrate, wherein W has one of the following structures: 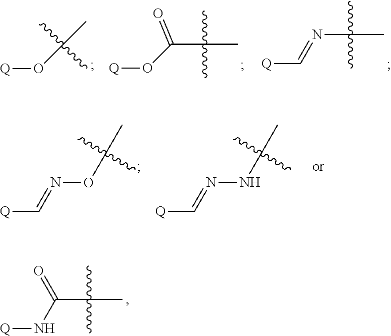
wherein Q is the outer surface of the substrate, and wherein the reactivity of the first and second thermochemically reactive groups are orthogonal to each other.

2. The solid support of claim 1, wherein the polymers have the following formula (I):
T¹-(A)ₓ(B)_{y}(C)_{z}-T² (I)
wherein:
A, B and C represent the A, B and C subunits, respectively;
T¹ and T² are each independently absent or polymer terminal groups selected from H, alkyl and an initiator residue;
x and z are independently an integer from 1 to 50,000; and
y is an integer from 0 to 50,000.

3. The solid support of claim 1, wherein the solid support has the following formula (II): 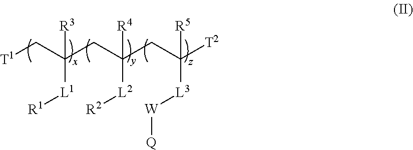
wherein:
R¹ is, at each occurrence, independently the first thermochemically reactive group, the second thermochemically reactive group or the covalent bond to the capture probe;
R² is, at each occurrence, independently the hydrophilic moiety;
W is, at each occurrence, independently the covalent bond to the outer surface of the substrate;
Q is the outer surface of the substrate;
R³, R⁴ and R⁵ are, at each occurrence, independently H or C₁-C₆ alkyl;
L¹, L² and L³ are, at each occurrence, independently a direct bond or a linker up to 100 atoms in length;
T¹ and T² are each independently absent or polymer terminal groups selected from H, alkyl and an initiator residue;
x and z are each independently an integer from 1 to 50,000; and
y is an integer from 0 to 50,000.

4. The solid support of claim 1, wherein at least one A subunit comprises a first thermochemically reactive group.

5. The solid support of claim 4, wherein the first thermochemically reactive group is an activated ester.

6. The solid support of claim 5, wherein the first thermochemically reactive group has, at each occurrence, independently the following formula: 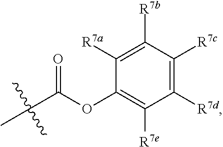
wherein R^{7a}, R^{7b}, R^{7c}, R^{7d} and R^{7e} are each independently H, halo, trihalomethyl, sulfo, —CN, C₁-C₆ alkyloxycarbonyl, C₁-C₆ hydroxylalkyloxycarbonyl, nitro or a polyethylene glycol moiety.

7-8. (canceled)

9. The solid support of claim 6, wherein each of R^{7a}, R^{7b}, R^{7c}, R^{7d} and R^{7e} are fluoro.

10. (canceled)

11. The solid support of claim 1, wherein at least one A subunit comprises the second thermochemically reactive group.

12-13. (canceled)

14. The solid support of claim 11, wherein the second thermochemically reactive group comprises a cycloaddition reactive group, and the cycloaddition reactive group comprises, at each occurrence, independently an alkyne or azide functional group.

15. The solid support of claim 14, wherein the cycloaddition reactive group has, at each occurrence, independently has one of the following formulas: 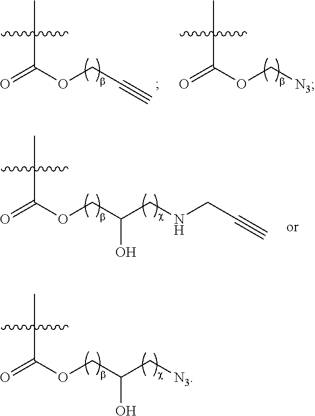
wherein β and χ are each independently integers ranging from 1 to 5.

16. The solid support of claim 15, wherein β is 1 or 3.

17. The solid support of claim 15, wherein χ is 1.

18. The solid support of claim 15, wherein the cycloaddition reactive group has, at each occurrence, independently one of the following formulas:

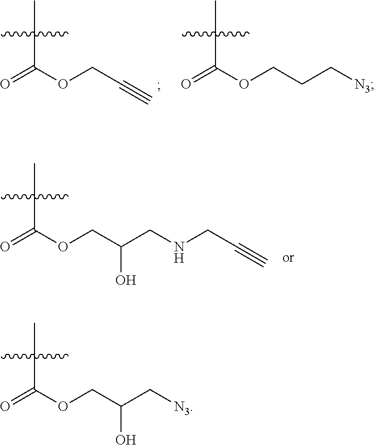

19-21. (canceled)

22. The solid support of claim 1, wherein at least one A subunit comprises a covalent bond to the capture probe.

23. The solid support of claim 22, wherein the covalent bond is an amidyl or amine bond to the capture probe.

24. (canceled)

25. The solid support of claim 22, wherein the at least one A subunit has one of the following structures: 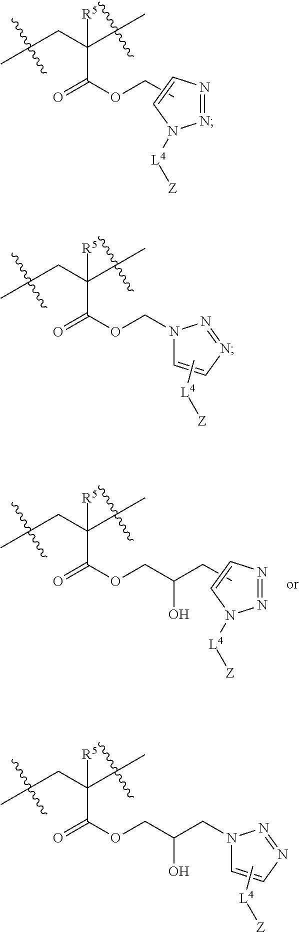
wherein:
R⁵ is H or C₁-C₆alkyl;
L⁴ is an optional linker; and
Z is the capture probe or fragment thereof.

26. The solid support of claim 1, wherein the capture probe is a peptide, protein, glycosylated protein, glycoconjugate, aptomer, carbohydrate, polynucleotide, oligonucleotide or polypeptide.

27. (canceled)

28. The solid support of claim 1, wherein the capture probe is DNA.

29. The solid support of claim 1, wherein W has, at each occurrence, independently one of the following structures: 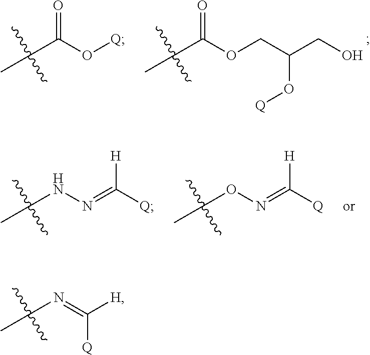
wherein Q is the outer surface of the solid substrate.

30. The solid support of claim 1, wherein the C subunit has, at each occurrence, independently one of the following structures: 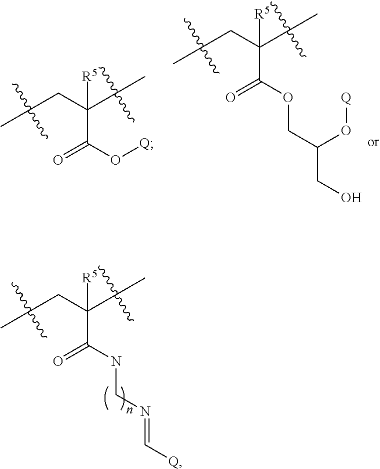
wherein:
R⁵ is, at each occurrence, independently H or C₁-C₆ alkyl;
Q is the outer surface of the solid support; and
n is an integer from 2 to 10.

31. The solid support of claim 1, wherein the hydrophilic moiety comprises, at each occurrence, independently amido, ester or hydroxyl functional groups, or combinations thereof.

32. The solid support of claim 1, wherein the polymer does not comprise B subunits.

33. The solid support of claim 1, wherein the polymer comprises one or more B subunit.

34. The solid support of claim 33, wherein the B subunit has, at each occurrence, independently one of the following formulas: 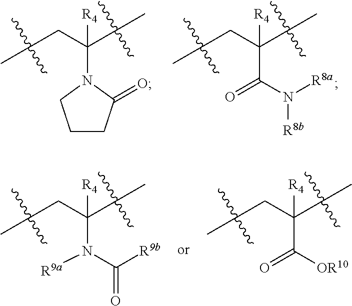
wherein:
R⁴ is at each occurrence, independently H or C₁-C₆ alkyl;
R^{8a} and R^{8b} are each independently H, C₁-C₆ alkyl or hydroxylalkyl;
R^{9a} and R^{9b} are each independently H, C₁-C₆ alkyl or hydroxylalkyl or R^{9a} and R^{9b}, together with the nitrogen atom to which they are bound, join to form a heterocyclic ring; and
R¹⁰ is hydroxylalkyl.

35-37. (canceled)

38. The solid support of claim 33, wherein each hydrophilic moiety has one of the following structures:

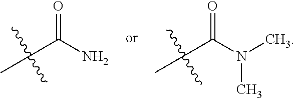

39-46. (canceled)

47. The solid support of claim 3, wherein each of L¹, L² and L³ are a direct bond.

48. (canceled)

49. The solid support of claim 3, wherein each of R³, R⁴ and R⁵ is H.

50-52. (canceled)

53. The solid support of claim 33, wherein the polymer comprises from greater than 0 mol % to about 40 mol % of B subunits.

54-56. (canceled)

57. The solid support of claim 33, wherein the sum of A and C subunits in the polymer comprises is about 60 mol %.

58-60. (canceled)

61. The solid support of claim 1, wherein each A subunit comprises the first thermochemically reactive group or the covalent bond to the capture probe.

62-64. (canceled)

65. The solid support of claim 1, wherein the solid support has a water contact angle ranging from 40° to 90°.

66-68. (canceled)

69. The solid support of claim 1, wherein the substrate comprises poly(styrene), poly(carbonate), poly(ethersulfone), poly(ketone), poly(aliphatic ether), poly(ether ketone), poly(ether ether ketone), poly(aryl ether), poly(amide) poly(imide), poly(ester) poly(acrylate), poly(methacrylate), poly(olefin), poly(cyclic olefin), poly(vinyl alcohol), polymer blends or poly alkyl polymers or halogenated derivatives, crosslinked derivatives or combinations thereof.

70. (canceled)

71. The solid support of claim 69, wherein the substrate comprises a cyclic poly(olefin).

72-73. (canceled)

74. The solid support of claim 1, wherein the substrate is at least about 90% optically transparent between about 400 nm and about 800 nm.

75. The solid support of claim 1, wherein the solid support comprises a systematic array of distinct locations, each distinct location independently comprising at least one of the polymers covalently bound to the outer surface of the substrate.

76. (canceled)

77. The solid support of claim 75, wherein at least one polymer at each distinct location independently comprises a capture probe covalently bound thereto.

78. (canceled)

79. The solid support of claim 1, wherein the plurality of polymers is substantially free of cross links therebetween.

80. A method for preparing the solid support of claim 1, the method comprising:
A) providing a solid substrate comprising a plurality of hydroxyl, carbonyl or amine functional groups, or combinations thereof covalently bound to the outer surface thereof, wherein the hydroxyl and carbonyl functional groups are bound directly to the solid substrate without intervening linkers, and the amine functional groups are bound to the solid support through a linker comprising an imine bond, the imine bond being bound directly to the solid substrate without an intervening linker; and
B) contacting a polymer comprising D and optional E and F subunits with the solid substrate under conditions sufficient to form a covalent bond between at least one of the hydroxyl, carbonyl or amine functional groups and the D subunit, wherein:
the D subunit, at each occurrence, independently comprises a first reactive group, wherein the first reactive group is a thermochemically reactive group capable of forming a covalent bond with an alcohol, carbonyl or amine functional group on a solid substrate or capture probe;
the E subunit, at each occurrence, independently comprises a hydrophilic moiety; and
the F subunit, at each occurrence, independently comprises a second reactive group, wherein the second reactive group is a cycloaddition or conjugate addition reactive group having a reactivity specific for covalent bond formation with a target functional group on a capture probe via a cycloaddition or 1,4-conjugate addition reaction,
wherein the reactivity of the first reactive group and the second reactive group are orthogonal to each other.

81-100. (canceled)

101. A method for determining the presence or absence of a target analyte molecule, the method comprising:
a) providing a solid support according to claim 1, wherein the A subunit comprises a capture probe covalently bound thereto;
b) contacting an analyte probe with the solid support; and
c) detecting the presence or absence of a signal produced from interaction of the capture probe with the analyte probe.

102-106. (canceled)

107. A solid substrate comprising a plurality of primary amine functional groups covalently bound to an outer surface of the solid support, wherein the amine functional groups are bound to the solid support through a linker comprising an imine bond.

108. (canceled)

109. A polymer comprising G, H and optional I subunits, wherein:
the G subunit, at each occurrence, independently comprises:
a) a first thermochemically reactive group, wherein the first thermochemically reactive group is capable of forming a covalent bond with an alcohol, carbonyl or amine group;
b) a second thermochemically reactive group, wherein the second thermochemically reactive group is a cycloaddition or conjugate addition reactive group having a reactivity specific for covalent bond formation with a target functional group via a cycloaddition or 1,4-conjugate addition reaction;
the H subunit, at each occurrence, has the following structure: 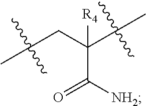
and
the optional I subunit, at each occurrence, independently comprises a hydrophilic moiety and has one of the following structures: 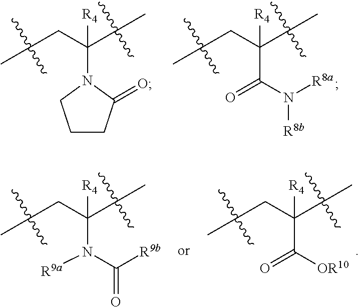
wherein:
R⁴ is at each occurrence, independently H or C₁-C₆ alkyl;
R^{8a} is H, C₁-C₆ alkyl or hydroxylalkyl;
R^{8b} is C₁-C₆ alkyl or hydroxylalkyl
R^{9a} and R^{9b} are each independently H, C₁-C₆ alkyl or hydroxylalkyl or R^{9a} and R^{9b}, together with the nitrogen atom to which they are bound, join to form a heterocyclic ring; and
R¹⁰ is hydroxylalkyl,
wherein the reactivity of the first and second thermochemically reactive groups are orthogonal to each other.

110-115. (canceled)

116. The solid support of claim 1, wherein the solid support has a water contact angle ranging from 61° to 95°.

117-119. (canceled)

120. The solid support of claim 1, wherein the solid support has a water contact angle of less than 15°.

121. (canceled)
